(19) United States
(12) Patent Application Publication
(10) Pub. No.: US 2026/0021119 A1
(43) Pub. Date: Jan. 22, 2026

(54) TETRACYCLINE DERIVATIVES

(71) Applicants: ICM (INSTITUT DU CERVEAU ET DE LA MOELLE ÉPINIÈRE), Paris (FR); INSERM (INSTITUT NATIONAL DE LA SANTÉ ET DE LA RECHERCHE MÉDICALE), Paris Cedex 13 (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITE, Paris (FR); UNIVERSITE PARIS-SACLAY, Gif-sur-Yvette (FR); UNIVERSIDADE DE SAO PAULO USP, São Paulo-SP (BR)

(72) Inventors: Bruno FIGADERE, Saint Chéron (FR); Laurent FERRIE, Palaiseau (FR); Alexis PINET, Paris (FR); Rita RAISMAN VOZARI, Paris (FR); Patrick Pierre MICHEL, Paris (FR); Elaine DEL-BEL, Sao Paulo (BR)

(73) Assignees: ICM (INSTITUT DU CERVEAU ET DE LA MOELLE ÉPINIÈRE), Paris (FR); INSERM (INSTITUT NATIONAL DE LA SANTÉ ET DE LA RECHERCHE MÉDICALE), Paris Cedex 13 (FR); CENTRE NATIONAL DE LA RECHERCHE SCIENTIFIQUE, Paris (FR); APHP (ASSISTANCE PUBLIQUE - HÔPITAUX DE PARIS), Paris (FR); SORBONNE UNIVERSITE, Paris (FR); UNIVERSITE PARIS-SACLAY, Gif-sur-Yvette (FR); UNIVERSIDADE DE SAO PAULO USP, São Paulo-SP (BR)

(21) Appl. No.: 18/881,402

(22) PCT Filed: Jul. 7, 2023
(86) PCT No.: PCT/EP2023/068918

§ 371 Date: Jan. 6, 2025

(30) Foreign Application Priority Data
Jul. 8, 2022 (EP) 22306025.2

(51) Int. Cl. A61K 31/65; A61P 25/28; A61P 29/00

(52) U.S. Cl. CPC A61K 31/65; A61P 25/28; A61P 29/00

(57) ABSTRACT

Tetracycline derivatives and their use as a medicament. In particular, the tetracycline derivatives may be used in the treatment or the prevention of a disease, such as amyloidosis, pain, neurodegenerative diseases and neuroinflammatory diseases, in which the tetracycline derivatives or pharmaceutical compositions including these compounds are administered to a subject in need thereof.

### FIELD OF INVENTION

The present invention relates to tetracycline derivatives and to their use as medicament in particular in the treatment or prevention of a disease selected from the group comprising or consisting of: amyloidosis, pain such as neuropathic pain, neuroinflammatory diseases and neurodegenerative diseases.

### BACKGROUND OF INVENTION

The physiopathology of many diseases is related to neurons, in particular by aggregation of proteins or by inflammation. For example, the aggregation of the protein alpha-synuclein into amyloid fibrils contributes to the pathogenesis of synucleinopathies, a group of neurodegenerative diseases comprising Parkinson's disease and other related disorders. Another example is the aggregation of the tau proteins into neurons, which contributes to the pathogenesis of Alzheimer's disease.

In the prior art, a wide range of tetracycline derivatives has been described for treating neurodegenerative diseases. For instance, WO 2004/064728 discloses specific tetracycline derivatives and their use for treating diseases or conditions involving underlying inflammatory processes, including among others, neurological disorders.

However, the antibacterial activity of tetracyclines represents a potential hurdle for the treatment of neurodegenerative diseases, because chronic treatments needed for these pathologies might favor the emergence of antibiotic-resistant pathogenic bacteria.

Thus, there remains a need to develop tetracycline derivatives having anti-protein-aggregation and anti-inflammatory properties and no antibiotic activity.

The inventors of the present invention have surprisingly discovered that tetracycline derivatives, which are compounds of formula (I) according to the present invention, have a better neuroprotection effect and/or anti-inflammatory effect and/or anti-aggregant activity than the tetracycline derivatives of the prior art, while having no antibiotic activity and being easy to manufacture, notably from doxycycline or oxytetracycline which possess a good safety profile.

### SUMMARY

The present invention relates to a compound of formula (I) or a pharmaceutically acceptable salt, solvate or mixtures thereof, wherein said formula (I) is as follows:

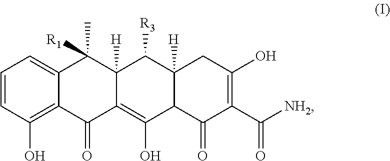

wherein R₁ and R₃ are each independently selected from the group consisting of a hydrogen atom and a hydroxyl group, for use in the treatment or prevention of a disease selected from the group consisting of: amyloidosis, pain, neurodegenerative diseases and neuroinflammatory diseases.

Advantageously, the compound of formula (I) is the compound of formula (Ibis) or a pharmaceutically acceptable salt, solvate or mixtures thereof, wherein said formula (Ibis) is as follows:

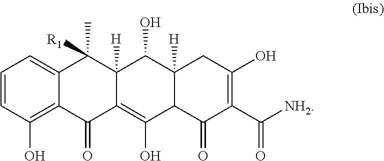

More advantageously, the compound of formula (I) is the compound of formula (Ia) or a pharmaceutically acceptable salt, solvate or mixtures thereof, wherein said formula (Ia) is as follows:

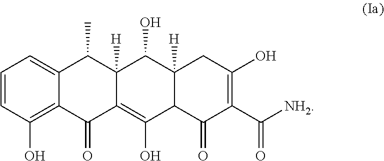 [SMILES: C[C@H]1c2cccc(O)c2C(=O)C2=C(O)C3C(=O)C(C(N)=O)=C(O)C[C@@H]3[C@@H](O)[C@@H]21]

More advantageously, the compound of formula (I) is the compound of formula (Ib) or a pharmaceutically acceptable salt, solvate or mixtures thereof, wherein said formula (Ib) is as follows:

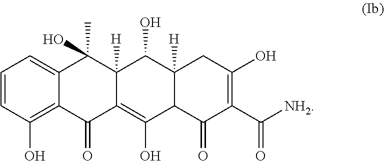 [SMILES: C[C@@]1(O)c2cccc(O)c2C(=O)C2=C(O)C3C(=O)C(C(N)=O)=C(O)C[C@@H]3[C@@H](O)[C@@H]21]

Advantageously, the compound of formula (I) is the compound of formula (Ic) or a pharmaceutically acceptable salt, solvate or mixtures thereof, wherein said formula (Ic) is as follows:

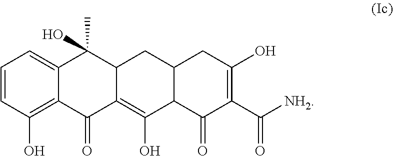 [SMILES: C[C@@]1(O)c2cccc(O)c2C(=O)C2=C(O)C3C(=O)C(C(N)=O)=C(O)CC3CC21]

Advantageously, said disease is selected from the group consisting of: amyloidosis, neuropathic pain, neurodegenerative diseases and neuroinflammatory diseases.

Advantageously, said neurodegenerative or neuroinflammatory diseases are selected from the group consisting of: Parkinson's disease, diffuse Lewy body disease, multiple system atrophy, Alzheimer's disease, prion disease, multiple sclerosis, limbic-predominant age-related TDP-43 encephalopathy (LATE), and Huntington's disease.

Advantageously, said neurodegenerative disease is Parkinson's disease or Alzheimer's disease, preferably said neurodegenerative disease is Parkinson's disease.

The present invention also relates to a method for manufacturing a compound of formula (I) or a pharmaceutically acceptable salt, solvate or mixtures thereof, as defined above, said method comprising the following steps:
a) reacting a compound of formula (II) or a pharmaceutically acceptable salt, solvate or mixtures thereof with a reducing system, wherein formula (II) is as follows:

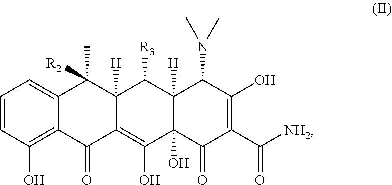

wherein R₂ and R₃ are each independently selected from the group consisting of a hydrogen atom or a hydroxyl group, and then
b) isolating the compound of formula (I) or the pharmaceutically acceptable salt, solvate or mixtures thereof, as defined above and produced during step a).

Advantageously, the reducing system in step a) comprises a reducing metal, preferably zinc metal, and optionally an acid, preferably acetic acid.

Advantageously, the reducing system in step a) is acid. More advantageously, the acidic reducing system in step a) comprises acetic acid.

Advantageously, the isolation of the compound of formula (I) or the pharmaceutically acceptable salt, solvate or mixtures thereof, as defined above, is made by chromatography, preferably by a chromatography selected from the group consisting of: column chromatography and reversed-phase high-performance liquid chromatography (reversed-phase HPLC).

The present invention also relates to a pharmaceutical composition comprising a compound of formula (I) or a pharmaceutically acceptable salt, solvate or mixtures thereof, as defined above, and at least one pharmaceutically acceptable excipient.

Advantageously, said at least one pharmaceutically acceptable excipient is selected from the group consisting of: microcrystalline cellulose, magnesium stearate, povidone, hydrogenated castor oil, colloidal anhydrous silica, sodium carboxymethyl starch and combinations thereof.

The present invention also relates to the pharmaceutical composition as defined above for use as a medicament.

The present invention further relates to the pharmaceutical composition as defined above for use in the treatment or prevention of a disease selected from the group consisting of: amyloidosis, pain, neurodegenerative disease and neuroinflammatory disease. Preferably, the neurodegenerative disease is selected from the group consisting of: Parkinson's disease, diffuse Lewy body disease, multiple system atrophy, Alzheimer's disease, prion disease, multiple sclerosis, limbic-predominant age-related TDP-43 encephalopathy (LATE), and Huntington's disease. More preferably, the neurodegenerative disease is Parkinson's disease.

The present invention further relates to the pharmaceutical composition as defined above for use in the treatment or prevention of a disease selected from the group consisting of: amyloidosis, neuropathic pain, neurodegenerative disease and neuroinflammatory disease. Preferably, the neurodegenerative disease is selected from the group consisting of: Parkinson's disease, diffuse Lewy body disease, multiple system atrophy, Alzheimer's disease, prion disease, multiple sclerosis, limbic-predominant age-related TDP-43 encephalopathy (LATE), and Huntington's disease. More preferably, the neurodegenerative disease is Parkinson's disease.

Advantageously, said pharmaceutical composition is administered by a route of administration selected from the following group consisting of: oral route, intraspinal route, intraarterial route, intravenous route, intramuscular route and subcutaneous route, preferably said pharmaceutical composition is administered by oral route.

The present invention further relates to a 12a-deoxytetracycline for use in the treatment of pain.

Advantageously, said 12a-deoxytetracycline is selected from the group consisting of the following compounds:

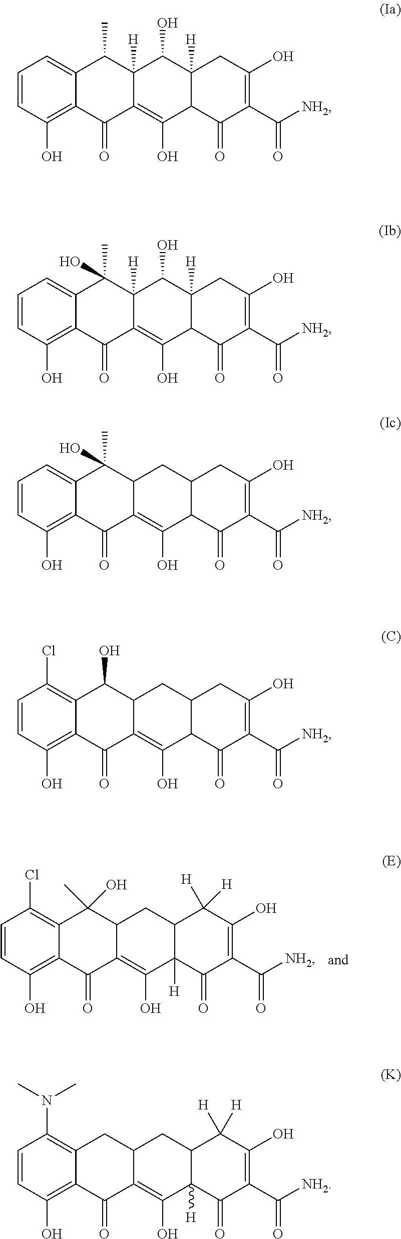 [SMILES: C[C@H]1c2cccc(O)c2C(=O)C2=C(O)C3C(=O)C(C(N)=O)=C(O)C[C@@H]3[C@@H](O)[C@@H]21 ; C[C@@]1(O)c2cccc(O)c2C(=O)C2=C(O)C3C(=O)C(C(N)=O)=C(O)C[C@@H]3[C@@H](O)[C@@H]21 ; C[C@@]1(O)c2cccc(O)c2C(=O)C2=C(O)C3C(=O)C(C(N)=O)=C(O)CC3CC21 ; NC(=O)C1=C(O)CC2CC3C(=C(O)C2C1=O)C(=O)c1c(O)ccc(Cl)c1[C@H]3O ; CC1(O)c2c(Cl)ccc(O)c2C(=O)C2=C(O)C3C(=O)C(C(N)=O)=C(O)CC3CC21 ; CN(C)c1ccc(O)c2c1CC1CC3CC(O)=C(C(N)=O)C(=O)C3C(O)=C1C2=O]

Advantageously, said pain is selected from the group consisting of: neuropathic pain, nociceptive pain or centralized pain.

More advantageously, said pain is a nociceptive pain induced by a disease selected from the group consisting of: osteoarthritis, rheumatoid arthritis and cancer, preferably by cancer.

More advantageously, said pain is a neuropathic pain induced by diabetic neuropathy.

More advantageously, said pain is a centralized pain induced by a disease selected from the group consisting of: fibromyalgia, irritable bowel syndrome or tension headaches.

### DEFINITIONS

In the present invention, the following terms have the following meanings:

“About”, before a figure or number, refers to plus or minus 10% of the face value of that figure or number. In one embodiment, “about”, before a figure or number, refers to plus or minus 5% of the face value of that figure or number.

“Active agent” refers to an agent that has a therapeutic effect. The agent may be a chemical or a biological substance. Preferably, the active agent is a chemical substance. The therapeutic effect may be the prevention, delay, reduction in severity and/or frequency or suppression of at least one symptom associated with a pathological condition, or the prevention, slowing down or suppression of the underlying cause of a pathological condition, or the improvement or repair of a damage.

“Alzheimer's disease” (AD) refers to a neurodegenerative disease that usually starts slowly and progressively worsens. It generally involves impairment of memory, judgment, attention span, and problem-solving skills and a global loss of cognitive abilities.

“Chlortetracycline” refers to the molecule of the following formula:

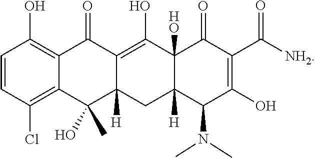 [SMILES: CN(C)[C@@H]1C(O)=C(C(N)=O)C(=O)[C@@]2(O)C(O)=C3C(=O)c4c(O)ccc(Cl)c4[C@@](C)(O)[C@H]3C[C@@H]12]

“Comprising” or “comprise” is to be construed in an open, inclusive sense, but not limited to. In an embodiment, “comprising” means “consisting essentially of”. In an embodiment, “comprising” means “consisting of”, which is to be construed as limited to. “Demeclocycline” refers to the molecule of the following formula:

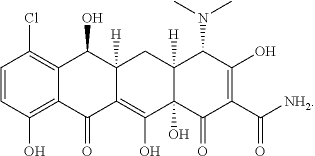 [SMILES: CN(C)[C@@H]1C(O)=C(C(N)=O)C(=O)[C@@]2(O)C(O)=C3C(=O)c4c(O)ccc(Cl)c4[C@@H](O)[C@H]3C[C@@H]12]

“Dose” refers to the amount of active agent administered at one time. Preferably, oral doses are administered to one subject from 8 hours to 1 week apart, preferably from 12 hours to 24 hours apart, more preferably about 24 hours apart. More preferably, said oral doses are human doses, wherein a human dose is a standard human dose for a man weighing 70 kg.

“Doxycycline” refers to the molecule of the following formula:

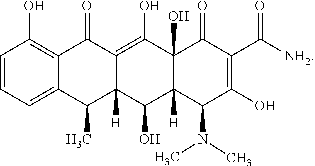 [SMILES: C[C@H]1c2cccc(O)c2C(=O)C2=C(O)[C@]3(O)C(=O)C(C(N)=O)=C(O)[C@@H](N(C)C)[C@@H]3[C@@H](O)[C@@H]21]

“Excipient” refers to any inactive ingredient, which is required for the formulation of an active agent in a suitable dosage form. In one embodiment, “excipient” refers to any and all solvents, diluent, carriers, fillers, bulking agents, binders, disintegrants, polymers, lubricants, glidants, surfactants, isotonic agents, thickening or emulsifying agents, stabilizers, absorption accelerators, flavoring agents, preservatives, antioxidants, buffering agents, gelling agents, solubilizing agents or any combination thereof.

“From X to Y” refers to the range of values between X and Y, the limits X and Y being included in said range.

“Neurodegenerative disease” refers to a disease or a disorder which involves a progressive damage of the structure or function of a neuron, including the death or demyelination of the neuron. According to the present invention, a neurodegenerative disease may also be a neuroinflammatory disease.

“Neuroinflammatory disease” refers to a disease or a disorder caused by an excessive inflammatory reaction of the nervous system.

“Neurodegenerative and/or neuroinflammatory diseases” refers to diseases or disorders that involves a progressive damage of the structure or function of a neuron, including the death or demyelination of the neuron, and/or that is caused by an excessive inflammatory reaction of the nervous system. Advantageously, neurodegenerative and/or neuroinflammatory diseases may be selected from the group comprising or consisting of Alzheimer's disease, dementia associated with Alzheimer's disease (e.g. Pick's disease), Parkinson's disease, diffuse Lewy body disease, senile dementia, Huntington's disease, encephalitis, Gilles de la Tourette syndrome, multiple sclerosis, amyotrophic lateral sclerosis (ALS), advanced supranuclear paralysis, epilepsy, schizophrenia, depression, post-traumatic stress disorder, Lou Gehrig's disease, Creutzfeldt-Jakob disease, stroke, fragile X syndrome, multiple system atrophy (MSA), pure autonomic dysfunction with synuclein deposition (PAF), hereditary neurodegeneration with iron accumulation in the brain, accidental Lewy body disease in the elderly, Lewy body subtype Alzheimer's disease, Down syndrome, progressive supranuclear palsy, essential tremor with Lewy bodies, familial parkinsonism with or without dementia, tau and progranulin gene-related dementia with or without parkinsonism, bovine spongiform encephalopathy, secondary Parkinson's disease, parkinsonism resulting from neurotoxin exposure, drug-induced parkinsonism with a-synuclein deposition, limbic-predominant age-related TDP-43 encephalopathy (LATE), and sporadic or hereditary spinocerebellar ataxia. Preferably, said neurodegenerative or neuroinflammatory disease is a synucleinopathy. More preferably, said neurodegenerative or neuroinflammatory disease is a synucleinopathy selected from the group comprising or consisting of Parkinson's disease, diffuse Lewy body disease, and multiple system atrophy. Even more preferably, said neurodegenerative or neuroinflammatory disease is Parkinson's disease.

“Neuropathic pain” refers to the pain initiated or caused by a primary lesion, dysfunction or transitory perturbation of the peripheral or central nervous system.

“Oxytetracycline” refers to the molecule of the following formula:

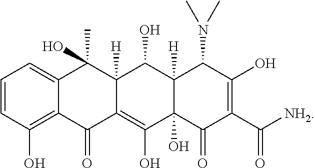 [SMILES: CN(C)[C@@H]1C(O)=C(C(N)=O)C(=O)[C@@]2(O)C(O)=C3C(=O)c4c(O)cccc4[C@@](C)(O)[C@H]3[C@H](O)[C@@H]12]

“Parkinson's disease” is a neurodegenerative disease that causes unintended or uncontrollable movements, such as shaking, stiffness, and difficulty with balance and coordination.

“Pharmaceutical composition” refers to a combination of at least one active agent and at least one pharmaceutically acceptable excipient.

“Pharmaceutically acceptable” refers to generally safe, non-toxic and neither biologically nor physiologically nor otherwise undesirable for mammalian animals, in particular for humans and non-human mammalian animals.

“Placebo” or “vehicle” refers to a pharmaceutical composition that comprises only pharmaceutically acceptable excipient(s), and no active agent.

“Prevent” or “preventing” refers to any action which makes it possible to prevent at least one symptom associated with a pathological condition, or to prevent the underlying cause of a pathological condition.

“Proteinopathy” refers to a class of diseases in which certain proteins become structurally abnormal, and thereby disrupt the function of cells, tissues and organs of the body. Examples of proteinopathies include, without limitation, Parkinson's disease, Huntington's disease, Alzheimer's disease and limbic-predominant age-related TDP-43 encephalopathy (LATE). In one embodiment, the proteinopathy is selected from the group comprising or consisting of Parkinson's disease, Huntington's disease, Alzheimer's disease and limbic-predominant age-related TDP-43 encephalopathy (LATE).

“Reducing system” refers to one or more reactants which are able to reduce a compound of formula (II) according to the present invention into a compound of formula (I) according to the present invention. Advantageously, the reducing system may comprise a reducing metal. The reducing metal may be a reducing transition metal such as, zinc, copper, iron, silver, gold, palladium, rhodium, iridium, platinum or nickel. Advantageously, the reducing metal is zinc metal. The reducing metal may be used, for instance, in the form of a metal powder. Advantageously, the reducing metal of the reducing system may be combined with an acid, for example selected from the group consisting of hydrochloric acid, hydrobromic acid, hydroiodic acid, sulfuric acid, nitric acid, phosphoric acid, formic acid, acetic acid, trifluoroacetic acid, methanesulfonic acid, benzenesulfonic acid, toluenesulfonic acid, and a mixture thereof. More advantageously, the reducing metal of the reducing system may be combined with acetic acid.

“Salt of a compound” refers to acid or base addition salts of said compound. The acid addition salts are formed with pharmaceutically acceptable organic or inorganic acids; the base addition salts are formed when an acid proton present in the compound is either replaced by a metal ion or coordinated with a pharmaceutically acceptable organic or inorganic base. In one embodiment, the acid addition salt is selected from the group consisting of acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. In one embodiment, the base addition salt is selected from the group consisting of aluminium, arginine, benzathine, calcium, choline, diethylamine, 2-(diethylamino) ethanol, diolamine, ethanolamine, glycine, 4-(2-hydroxyethyl)-morpholine, lysine, magnesium, meglumine, morpholine, olamine, potassium, sodium, tromethamine and zinc salts.

“Solvate of a compound” refers to a molecular complex comprising said compound and one or more pharmaceutically acceptable solvent molecules. “Hydrate of a compound” refers to a molecular complex comprising the compound and one or more pharmaceutically acceptable solvent molecules, wherein the solvent is water.

“Subject” or “patient” refers to a mammalian animal, wherein “mammalian animal” refers to a human or a non-human mammalian animal. Preferably, “subject” or “patient” refers to a human (man or woman). More preferably, “subject” or “patient” refers to a human of at least 18 years old, advantageously of at least 40 years old, more advantageously of at least 50 years old, still more advantageously of at least 60 years old, even more advantageously of at least 70 years old or at least 80 years old.

“Synucleinopathy” refers to a neurodegenerative disease and/or a neuroinflammatory disease characterized by an abnormal accumulation of aggregates of alpha-synuclein protein in neurons, nerve fibers and/or glial cells. Advantageously, synucleinopathy may be selected from the group comprising or consisting of Parkinson's disease, diffuse Lewy body disease (DLB), multiple system atrophy (MSA), pure autonomic dysfunction with synuclein deposition (PAF), hereditary neurodegeneration with iron accumulation in the brain, accidental Lewy body disease in the elderly, Lewy body subtype Alzheimer's disease, Down syndrome, essential tremor with Lewy bodies, familial parkinsonism with or without dementia, tau and progranulin gene-related dementia with or without parkinsonism, Creutzfeldt-Jakob disease, bovine spongiform encephalopathy, secondary Parkinson's disease, parkinsonism resulting from neurotoxin exposure, drug-induced parkinsonism with a-synuclein deposition, sporadic or hereditary spinocerebellar ataxia, and amyotrophic lateral sclerosis (ALS).

“Tetracycline” refers to the molecule of the following formula:

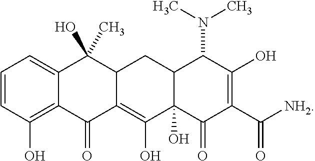 [SMILES: CN(C)[C@@H]1C(O)=C(C(N)=O)C(=O)[C@@]2(O)C(O)=C3C(=O)c4c(O)cccc4[C@@](C)(O)C3CC12]

with the following numeration of the atoms:

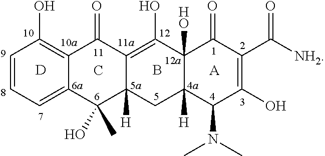

“Tetracycline derivative” refers to molecules having formula closed to the formula of tetracycline, in particular with a backbone of 4 fused rings but with at least one chemical group differing from the formula of tetracycline, for example a molecule whose chemical formula differs from tetracycline in 1 to 6 chemical groups, preferably in 2 to 4 chemical groups.

“12a-deoxytetracycline” refers to tetracycline derivatives having chemical formulas in which the hydroxyl group linked to carbon number 12a of the tetracycline formula is replaced by a hydrogen atom.

“Therapeutically effective amount” or “effective amount” of an active agent or of a composition refers to a nontoxic but sufficient amount of said active agent or composition to provide the desired therapeutic effect.

“Treating” or “treatment” refers to any action which makes it possible to delay, reduce in severity and/or frequency or suppress at least one symptom associated with a pathological condition, or to slow down or suppress the underlying cause of a pathological condition, or the improvement or remediation of damage. In one embodiment, “treatment” refers to a curative treatment.

### DETAILED DESCRIPTION

### Compound of formula (I)

This invention describes a compound of formula (I) or a pharmaceutically acceptable salt, solvate or mixtures thereof, wherein said formula (I) is:

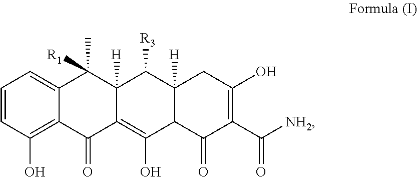

wherein R₁ and R₃ are each independently selected from the group consisting of a hydrogen atom and a hydroxyl group.

Advantageously, the compound of formula (I) is the compound of formula (Ibis) or a pharmaceutically acceptable salt, solvate or mixtures thereof, wherein said formula (Ibis) is as follows:

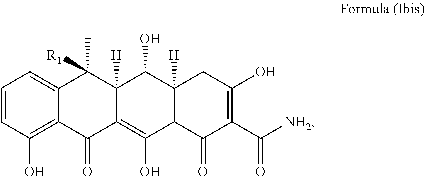

wherein R₁ is a hydrogen atom or a hydroxyl group.

More advantageously, the compound of formula (Ibis) is selected from the group consisting of the compound of formula (Ia) and the compound of formula (Ib), wherein formula (Ia) is as follows:

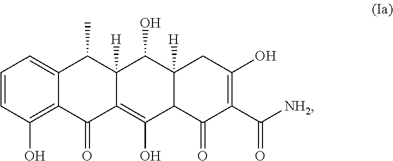 [SMILES: C[C@H]1c2cccc(O)c2C(=O)C2=C(O)C3C(=O)C(C(N)=O)=C(O)C[C@@H]3[C@@H](O)[C@@H]21]

and formula (Ib) is as follows:

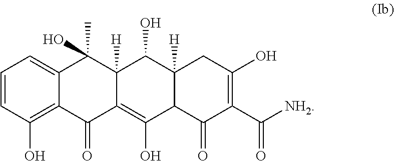 [SMILES: C[C@@]1(O)c2cccc(O)c2C(=O)C2=C(O)C3C(=O)C(C(N)=O)=C(O)C[C@@H]3[C@@H](O)[C@@H]21]

More advantageously, the compound of formula (I) is the compound of formula (Ia). The chemical name of the compound of formula (Ia) is (4aS,5R,5aR,6R)-3,5,10,12-tetrahydroxy-6-methyl-1,11-dioxo-1,4,4a,5,5a,6,11,12a-octahydrotetracene-2-carboxamide (named using ChemBioDraw® Ultra version 12.0 (PerkinElmer)).

More advantageously, the compound of formula (I) is the compound of formula (Ib). The chemical name of the compound of formula (Ib) is (4aS,5R,5aR,6S)-3,5,6,10,12-pentahydroxy-6-methyl-1,11-dioxo-1,4,4a,5,5a,6,11,12a-octahydrotetracene-2-carboxamide (named using ChemBioDraw® Ultra version 12.0 (PerkinElmer)).

Advantageously, the compound of formula (I) is the compound of formula (Ic) or a pharmaceutically acceptable salt, solvate or mixtures thereof, wherein said formula (Ic) is as follows:

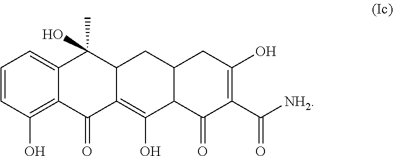 [SMILES: C[C@@]1(O)c2cccc(O)c2C(=O)C2=C(O)C3C(=O)C(C(N)=O)=C(O)CC3CC21]

### Compound of Formula (I) for Use as a Medicament

The present invention also describes a compound of formula (I) or a pharmaceutically acceptable salt, solvate or mixtures thereof for use as a medicament in a subject in need thereof.

The present invention also describes a method of preventing and/or treating a disease by administering to a subject in need thereof an effective amount of a compound of formula (I) or a pharmaceutically acceptable salt, solvate or mixtures thereof.

The present invention also describes the use of a compound of formula (I) or a pharmaceutically acceptable salt, solvate or mixtures thereof for the manufacture of a medicament.

The present invention also describes the use of a compound of formula (I) or a pharmaceutically acceptable salt, solvate or mixtures thereof for the prevention and/or treatment of a disease in a subject.

### Compound of Formula (I) for Use in the Treatment of Diseases

The present invention relates to a compound of formula (I) or a pharmaceutically acceptable salt, solvate or mixtures thereof according to the present application, for use in the prevention and/or treatment of a disease selected from the group comprising or consisting of: amyloidosis, pain (preferably neuropathic pain), neurodegenerative diseases and neuroinflammatory diseases.

The present invention also relates to a method of preventing and/or treating a disease selected from the group comprising or consisting of: amyloidosis, pain (preferably neuropathic pain), neurodegenerative diseases and neuroinflammatory diseases, by administering to a subject in need thereof an effective amount of a compound of formula

(I) or a pharmaceutically acceptable salt, solvate or mixtures thereof according to the present application.

The present invention also relates to the use of a compound of formula (I) or a pharmaceutically acceptable salt, solvate or mixtures thereof according to the present application for the manufacture of a medicament for the prevention and/or treatment in a subject of a disease selected from the group comprising or consisting of: amyloidosis, pain (preferably neuropathic pain), neurodegenerative diseases and neuroinflammatory diseases.

The present invention also relates to the use of a compound of formula (I) or a pharmaceutically acceptable salt, solvate or mixtures thereof according to the present application for the prevention and/or treatment in a subject of a disease selected from the group comprising or consisting of: amyloidosis, pain (preferably neuropathic pain), neurodegenerative diseases and neuroinflammatory diseases.

Advantageously, the compound of formula (I) is the compound of formula (Ibis) according to the invention.

More advantageously, the compound of formula (I) is the compound of formula (Ia) according to the invention.

More advantageously, the compound of formula (I) is the compound of formula (Ib) according to the invention.

Advantageously, the compound of formula (I) is the compound of formula (Ic) according to the invention.

In one embodiment, the disease is pain. Preferably, said pain is selected from the group consisting of: neuropathic pain, nociceptive pain or centralized pain. More preferably, said pain is a nociceptive pain. Even more preferably, said pain is a nociceptive pain induced by a disease selected from the group consisting of: osteoarthritis, rheumatoid arthritis and cancer, preferably by cancer. More preferably, said pain is a neuropathic pain induced by diabetic neuropathy. More preferably, said pain is a centralized pain induced by a disease selected from the group consisting of: fibromyalgia, irritable bowel syndrome or tension headaches.

In one embodiment, the disease is a neurodegenerative and/or neuroinflammatory disease.

In one embodiment, the disease is a proteinopathy.

Advantageously, the neurodegenerative or neuroinflammatory diseases are selected from the group consisting of: Parkinson's disease, diffuse Lewy body disease, multiple system atrophy, Alzheimer's disease, prion disease, multiple sclerosis, limbic-predominant age-related TDP-43 encephalopathy (LATE), and Huntington's disease.

More advantageously, the neurodegenerative diseases are selected from the group consisting of: Parkinson's disease and Alzheimer's disease.

In one embodiment, the neurodegenerative disease is Parkinson's disease.

In one embodiment, the neurodegenerative disease is Alzheimer's disease.

In one embodiment, the neurodegenerative disease is limbic-predominant age-related TDP-43 encephalopathy (LATE).

In one embodiment, the disease is a neuroinflammatory disease. Said inflammatory disease may be caused by various causes, including, for example, toxic compounds, auto-immunity, ageing, infectious agents such as viruses, bacteria or parasites, nervous system injuries, and pollution.

In one embodiment, the neuroinflammatory disease is caused by a virus. Examples of virus that can cause neuroinflammatory disease include, for example, encephalitis viruses, human immunodeficiency viruses, polioviruses, and coronaviruses.

In one embodiment, the neuroinflammatory disease is caused by a coronavirus, such as severe acute respiratory syndrome coronavirus 2 (SARS-Cov-2). Thus, in one embodiment, the neuroinflammatory disease is COVID-19.

In one embodiment, the subject in need thereof is a subject in which a disease as defined hereinabove is to be prevented.

In one embodiment, the subject in need thereof is affected, preferably diagnosed, with a disease as defined hereinabove.

In one embodiment, the subject is presymptomatic, meaning that said subject does not present symptoms of the disease. In one embodiment, the subject is symptomatic, meaning that said subject present with at least one symptom of the disease.

In one embodiment, the subject in need thereof is affected, preferably diagnosed, with Parkinson's disease.

Examples of symptoms of Parkinson's disease include, without limitation, tremor in hands, arms, legs, jaw, or head, muscle stiffness, where muscle remains contracted for a long time, slowness of movement, or impaired balance and coordination, sometimes leading to falls.

Methods for diagnosing Parkinson's disease are well known by the skilled artisan in the art, and include, for example, neurological examination and tests such as the Unified Parkinson's Disease Rating Scale (UPDRS), and dopamine transporter scan (DaTscan).

In one embodiment, the subject in need thereof is affected, preferably diagnosed, with Alzheimer's disease.

Examples of symptoms of Alzheimer's disease include, without limitation, confusion, disorientation, difficulty planning or making decisions, problems with speech and language, personality changes and low mood or anxiety

Methods for diagnosing Alzheimer's disease are well known by the skilled artisan in the art, and include, for example, physical, neurological and mental exams.

In one embodiment, the subject in need thereof is affected, preferably diagnosed, with neuropathic pain.

Examples of symptoms of neuropathic pain include, without limitation, spontaneous pain, evoked pain, pain that may be lessened in response to a normally painful stimulus.

### Method for Manufacturing Compounds of Formula (I)

The present invention also relates to a method for manufacturing a compound of formula (I) or a pharmaceutically acceptable salt, solvate or mixtures thereof according to the present invention.

Said method comprises the following successive steps
a) reacting a compound of formula (II) or a pharmaceutically acceptable salt, solvate or mixtures thereof, with a reducing system, wherein formula (II) is as follows:

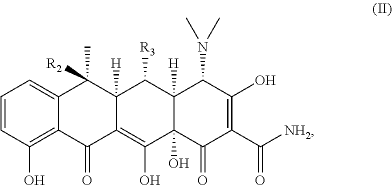

wherein R₂ and R₃ are each independently selected from the group consisting of a hydrogen atom or a hydroxyl group, and then
b) isolating a compound of formula (I), or a pharmaceutically acceptable salt, solvate or mixtures thereof, which has been produced during step a).

Preferably, said method comprises the following successive steps:
a) reacting a compound of formula (IIbis) or a pharmaceutically acceptable salt, solvate or mixtures thereof, with a reducing system, wherein formula (IIbis) is as follows:

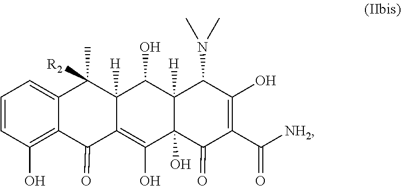

wherein R₂ is a hydrogen atom or a hydroxyl group, and then
b) isolating a compound of formula (I), or a pharmaceutically acceptable salt, solvate or mixtures thereof, which has been produced during step a).

Advantageously, the compound of formula (I) is selected from the group consisting of the compounds of formula (Ibis) and (Ic) according to the present application.

Advantageously, the compound of formula (I) is selected from the group consisting of the compounds of formula (Ia), (Ib) and (Ic) according to the present application.

Advantageously, the compound of formula (I) is selected from the group consisting of the compound of formula (Ia) according to the invention and the compound of formula (Ib) according to the present application.

Advantageously, the compound of formula (I) is the compound of formula (Ia) according to the present application.

Advantageously, the compound of formula (I) is the compound of formula (Ib) according to the present application.

Advantageously, the compound of formula (I) is the compound of formula (Ic) according to the present application.

Advantageously, R₂ is a hydrogen atom.

Advantageously, the compound of formula (II) is doxycycline, preferably doxycycline hydrochloride.

Advantageously, R₂ is a hydroxyl group.

Advantageously, the compound of formula (II) is oxytetracycline.

Advantageously, the compound of formula (I) is the compound of formula (Ia) according to the invention and the compound of formula (II) is doxycycline, preferably doxycycline hydrochloride.

Advantageously, the compound of formula (I) is the compound of formula (Ib) according to the invention and the compound of formula (II) is oxytetracycline.

Step a) is performed before step b).

Advantageously, the reducing system in step a) comprises a reducing metal, preferably zinc metal.

Advantageously, the reducing system in step a) is acid. More advantageously, the acidic reducing system in step a) comprises acetic acid. Indeed, the inventors of the present invention have surprisingly discovered that an acidic reducing system allows the obtention of compounds of formula (I) in the majority and preferably obtained. In addition, when the reducing system in step a) is acid, there is only one step of reduction in the process, said step enabling the double reduction of the compound of formula (II) and the obtention of a compound of formula (I).

Advantageously, the reducing system in step a) comprises an acid, such as acetic acid.

More advantageously, the reducing system in step a) comprises a reducing metal and an acid, preferably zinc metal and acetic acid.

Advantageously, the amount of reducing metal may be catalytic, stoichiometric, or in excess, preferably in excess, with respect to the compound of formula (II). In particular, the amount of reducing metal may be comprised between 3 and 30 equivalents, preferably between 5 and 15 equivalents, with respect to the compound of formula (II).

Advantageously, step a) may be carried out at a temperature ranging from 0° C. to 50° C., preferably from 15° C. to 35° C., more preferably of about 25° C.

Advantageously, step a) may be carried out during at least 2 hours, preferably during at least 4 hours, more preferably during about 8 hours.

Advantageously, step a) may be carried out at a pressure of 1 atm.

Advantageously, in step b), the isolation of the compound of formula (I), or a pharmaceutically acceptable salt, solvate or mixtures thereof, is made by chromatography. Preferably, the chromatography is selected from the group consisting of: column chromatography and reversed-phase high-performance liquid chromatography (reversed-phase HPLC). More preferably, the chromatography is a column chromatography.

### Pharmaceutical Composition

The invention also relates to a pharmaceutical composition comprising a compound of formula (I) or a pharmaceutically acceptable salt, solvate or mixtures thereof according to the present invention, and at least one pharmaceutically acceptable excipient.

Advantageously, the compound of formula (I) is selected from the group consisting of the compounds of formula (Ibis) and (Ic) according to the present application.

Advantageously, the compound of formula (I) is selected from the group consisting of the compounds of formula (Ia), (Ib) and (Ic) according to the present application.

Advantageously, the compound of formula (I) is the compound of formula (Ibis) according to the invention.

More advantageously, the compound of formula (I) is the compound of formula (Ia) according to the invention.

More advantageously, the compound of formula (I) is the compound of formula (Ib) according to the invention.

Advantageously, the compound of formula (I) is the compound of formula (Ic) according to the invention.

Advantageously, the pharmaceutical composition comprises from 0.1% to 99% by weight, preferably from 1% to 50% by weight, more preferably from 5% to 25% by weight, even more preferably about 20% by weight, of a compound of formula (I) or a pharmaceutically acceptable salt, solvate or mixtures thereof according to the present invention, relative to the total weight of said pharmaceutical composition.

Advantageously, for oral administration, the pharmaceutical composition may be formulated into conventional oral dosage forms such as tablets, gels, capsules, powders, granules and liquid preparations such as syrups, elixirs, and concentrated drops. Nontoxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

Advantageously, the at least one pharmaceutically acceptable excipient of the pharmaceutical composition is selected from the group consisting of: microcrystalline cellulose, magnesium stearate, povidone, hydrogenated castor oil, colloidal anhydrous silica, sodium carboxymethyl starch and combinations thereof.

Advantageously, the pharmaceutical composition may be formulated to release the compound of formula (I) or a pharmaceutically acceptable salt, solvate or mixtures thereof according to the present invention substantially immediately upon administration or at any predetermined time or time period after administration.

### Pharmaceutical Composition for Use

The invention also relates to the pharmaceutical composition according to the invention as described above for use as a medicament.

The present invention also relates to a method of preventing and/or treating a disease by administering to a subject in need thereof an effective amount of the pharmaceutical composition according to the invention as described above.

The present invention also relates to the use of the pharmaceutical composition according to the invention as described above for the manufacture of a medicament.

The present invention also relates to the use of the pharmaceutical composition according to the invention as described above for the prevention and/or treatment of a disease in a subject.

The invention also relates to the pharmaceutical composition according to the invention as described above for use in the prevention and/or treatment of a disease selected from the group comprising or consisting of: amyloidosis, pain, neurodegenerative diseases and neuroinflammatory diseases.

The invention also relates to the pharmaceutical composition according to the invention as described above for use in the prevention and/or treatment of a disease selected from the group comprising or consisting of: amyloidosis, neuropathic pain, neurodegenerative diseases and neuroinflammatory diseases.

The present invention also relates to a method of preventing and/or treating a disease selected from the group comprising or consisting of: amyloidosis, neuropathic pain, neurodegenerative diseases and neuroinflammatory diseases, by administering to a subject in need thereof an effective amount of the pharmaceutical composition according to the invention as described above.

The present invention also relates to the use of the pharmaceutical composition according to the invention as described above for the manufacture of a medicament for the prevention and/or treatment in a subject of a disease selected from the group comprising or consisting of: amyloidosis, neuropathic pain, neurodegenerative diseases and neuroinflammatory diseases.

The present invention also relates to the use of the pharmaceutical composition according to the invention as described above for the prevention and/or treatment in a subject of a disease selected from the group comprising or consisting of: amyloidosis, neuropathic pain, neurodegenerative diseases and neuroinflammatory diseases.

In one embodiment, the disease is pain. Preferably, said pain is selected from the group consisting of: neuropathic pain, nociceptive pain or centralized pain. More preferably, said pain is a nociceptive pain. Even more preferably, said pain is a nociceptive pain induced by a disease selected from the group consisting of: osteoarthritis, rheumatoid arthritis and cancer, preferably by cancer. More preferably, said pain is a neuropathic pain induced by diabetic neuropathy. More preferably, said pain is a centralized pain induced by a disease selected from the group consisting of: fibromyalgia, irritable bowel syndrome or tension headaches.

The advantageous features described in part “Compound of formula (I) for use in the treatment of diseases” of the present application apply mutatis mutandis for the pharmaceutical composition for use in the prevention and/or treatment of a disease selected from the group comprising or consisting of: amyloidosis, neuropathic pain, neurodegenerative diseases and neuroinflammatory diseases.

Route of administration and dose

Advantageously, the compound of formula (I) or a pharmaceutically acceptable salt, solvate or mixtures thereof according to the invention or the pharmaceutical composition according to the invention is administered by enteral or parenteral route of administration.

Advantageously, the compound of formula (I) is selected from the group consisting of the compounds of formula (Ibis) and (Ic) according to the present application.

Advantageously, the compound of formula (I) is selected from the group consisting of the compounds of formula (Ia), (Ib) and (Ic) according to the present application.

Advantageously, the compound of formula (I) is the compound of formula (Ibis) according to the invention.

More advantageously, the compound of formula (I) is the compound of formula (Ia) according to the invention.

More advantageously, the compound of formula (I) is the compound of formula (Ib) according to the invention.

Advantageously, the compound of formula (I) is the compound of formula (Ic) according to the invention.

More advantageously, the enteral route may be selected from the group consisting of: buccal route (including perlingual route and sublingual route), oral route and rectal route. Preferably, the compound of formula (I) or a pharmaceutically acceptable salt, solvate or mixtures thereof according to the invention or the pharmaceutical composition according to the invention is administered by oral route.

The parenteral includes any route that is not enteral. More advantageously, the parenteral route may be selected from the group consisting of: epidural route, intraspinal route, intracerebral route, intracerebroventricular route, epicutaneous route, transdermal route, intradermal route, subcutaneous route, nasal route, intra-arterial route, intra-articular route, intravenous route, intramuscular route, intraperitoneal route, intraocular route, intravitreal route, intrathecal route and intravitreal route. Preferably, the compound of formula (I) or a pharmaceutically acceptable salt, solvate or mixtures thereof according to the invention or the pharmaceutical composition according to the invention is administered by intravenous route.

Advantageously, one dose of the compound of formula (I) or a pharmaceutically acceptable salt, solvate or mixtures thereof according to the invention or the pharmaceutical composition according to the invention is administered regularly, preferably from three times per day to one time per month, more preferably from three times per day to one time per week, even more preferably from three times per day to one time per day. More advantageously, one dose of the compound of formula (I) or a pharmaceutically acceptable salt, solvate or mixtures thereof according to the invention or the pharmaceutical composition according to the invention is administered one, two or three times per day, preferably one time per day.

Advantageously, the dose of the compound of formula (I) or pharmaceutically acceptable salt, solvate or mixtures thereof according to the invention which is administered to the subject is from about 1 to 100 mg/kg body weight of the subject by intravenous, intramuscular injection or oral route, preferably by oral route.

Advantageously, the dose of the compound of formula (I) or pharmaceutically acceptable salt, solvate or mixtures thereof according to the invention which is administered to the subject ranges from 50 mg to 150 mg, preferably from 70 mg to 130 mg, more preferably the dose is of about 100 mg.

12a-deoxytetracycline for Use in the Treatment of Pain

The present invention also relates to 12a-deoxytetracycline for use in the treatment of pain.

The present invention also relates to a method of preventing and/or treating pain by administering to a subject in need thereof an effective amount of a 12a-deoxytetracycline.

The present invention also relates to the use of a 12a-deoxytetracycline for the manufacture of a medicament for the prevention and/or treatment of pain in a subject in need thereof.

The present invention also relates to the use of a 12a-deoxytetracycline for the prevention and/or treatment of pain in a subject in need thereof.

Advantageously, the 12a-deoxytetracycline is selected from the group consisting of the following compounds:

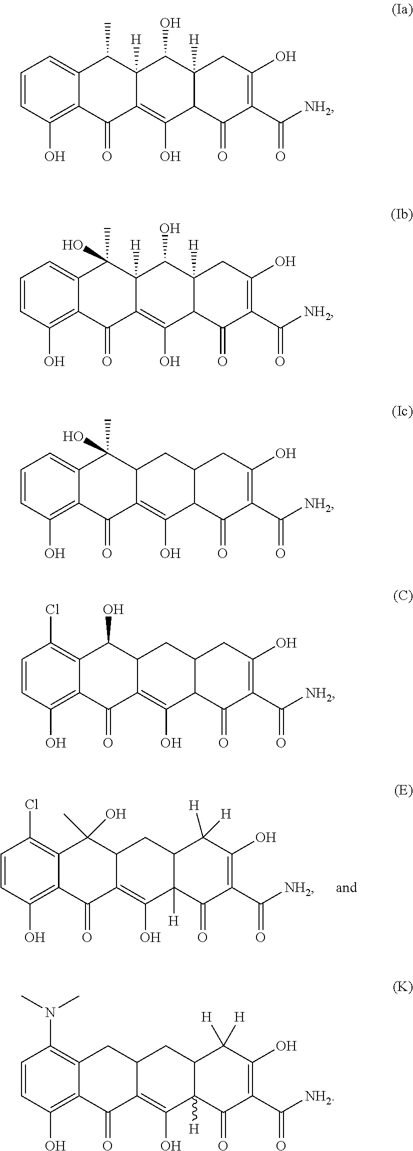 [SMILES: C[C@H]1c2cccc(O)c2C(=O)C2=C(O)C3C(=O)C(C(N)=O)=C(O)C[C@@H]3[C@@H](O)[C@@H]21 ; C[C@@]1(O)c2cccc(O)c2C(=O)C2=C(O)C3C(=O)C(C(N)=O)=C(O)C[C@@H]3[C@@H](O)[C@@H]21 ; C[C@@]1(O)c2cccc(O)c2C(=O)C2=C(O)C3C(=O)C(C(N)=O)=C(O)CC3CC21 ; NC(=O)C1=C(O)CC2CC3C(=C(O)C2C1=O)C(=O)c1c(O)ccc(Cl)c1[C@H]3O ; CC1(O)c2c(Cl)ccc(O)c2C(=O)C2=C(O)C3C(=O)C(C(N)=O)=C(O)CC3CC21 ; CN(C)c1ccc(O)c2c1CC1CC3CC(O)=C(C(N)=O)C(=O)C3C(O)=C1C2=O]

Indeed, as demonstrated in Example 10 of the present application, 12a-deoxytetracycline have anti-nociceptive effects.

Advantageously, said pain is selected from the group consisting of: neuropathic pain, nociceptive pain or centralized pain.

More advantageously, said pain is a nociceptive pain induced by a disease selected from the group consisting of: osteoarthritis, rheumatoid arthritis and cancer, preferably by cancer.

More advantageously, said pain is a neuropathic pain induced by diabetic neuropathy.

More advantageously, said pain is a centralized pain induced by a disease selected from the group consisting of: fibromyalgia, irritable bowel syndrome or tension headaches.

Advantageously, the 12a-deoxytetracycline is the compound of formula (Ia) and the subject is a female or woman.

Advantageously, the 12a-deoxytetracycline is the compound of formula (C) and the subject is a male or man.

Indeed, as demonstrated in Example 10 of the present application, there is a sex-dependent analgesic effect of the compound of formula (C) and the compound of formula (Ia).

### BRIEF DESCRIPTION OF THE DRAWINGS

FIG. 1 is a histogram showing the alpha-synuclein aggregation as a function of the concentration of the compound of formula (Ia) according to the invention (FIG. 1A) and two TEM pictures of alpha-synuclein fibrils, in the absence and in the presence of the compound of formula (Ia) according to the invention (FIG. 1B). In FIG. 1A: Fluorescence emission intensity of thioflavin T (ThT) in a solution containing 70 μM alpha-synuclein, in the presence of 0 μM (aS=control), 5 μM, 10 μM, 20 μM or 50 μM of the compound of formula (Ia) according to the invention after 120 hours, at 37° C., of incubation. Statistical analyses were performed with: Kruskal-Wallis test, * p<0.05; ** p<0.01; *** p<0.0001 vs. αS. (n=3). Bars=Mean+SEM. In FIG. 1B: TEM pictures of alpha-synuclein samples incubated at 37° C. under orbital agitation for 5 days in absence (top picture “αS”) and in presence (bottom picture “aS: DR2 20 μM”) of 20 μM of the compound of formula (Ia) according to the invention. Magnification corresponds to 44000X. Scale bar =1 μm.

FIG. 2 is a histogram showing the neuroprotective effect of 10 μM of doxycycline (DOX) and of 3 μM and 5 μM of the compound of formula (Ia) according to the present invention, compared with the neuroprotective effect of 100 μg/mL of apotransferrin (APO) (used as a positive control). Values are represented as the mean±SEM. One-way ANOVA followed by Dunnett's multiple comparisons test (n=6-8). *** p<0.0001 vs Control (-); Doxycycline (DOX; 10 μM); Compound of formula (Ia) was used at 3 μM or 5 μM; APO (Apotransferrin; 100 μg/mL).

FIG. 3 is a histogram showing the anti-inflammatory effect of 5 μM and 20 μM of compound or formula (B) presented in Example 1, of 5 μM and 20 μM of compound of formula (Ia) according to the present invention and of 50 μM of doxycycline (DOX), compared to the effect of 2.5 μM of dexamethasone (DEX) (used as a positive control). Values are represented as the mean±SEM. Mann-Whitney, u-test (n=4). **p=0.01; ***p=0.004 vs. LPS; Untreated (-), Dexamethasone (DEX; 2.5 μM); Doxycycline (DOX; 50 μM).

FIG. 4 is a histogram showing the anti-inflammatory effect of 10 μM and 20 μM of compound of formula (C) and of 10 μM and 20 μM of compound of formula (Ia) according to the present invention, compared to the effect of 2.5 μM of dexamethasone (DEX) (used as a positive control). The condition “NT” represents the control condition in the absence of treatment by LPS. Values are represented as the mean±SEM. Dunnett's multiple comparisons test (n=4). ** p=0.0008; **** p<0.0001 vs. LPS; Untreated (NT); Dexamethasone (DEX; 2.5 μM).

FIG. 5 is a histogram showing the neuroprotective effect of 10 μM of doxycycline (DOX) and of 0.5 μM, 1 μM and 5 μM of the compound of formula (Ib) according to the present invention, compared with the neuroprotective effect of 100 μg/mL of apotransferrin (APO) (used as a positive control). Data expressed in % of APO-treated cultures are presented as mean±SEM (n=4-16). ANOVA followed by Dunnett's post-hoc test. *** p<0.001 vs non-treated cultures.

FIG. 6 is a histogram showing the neuroprotective effect of 0.5 μM, 1 μM and 5 μM of the compound of formula (Ic) according to the present invention, compared with the neuroprotective effect of 100 μg/mL of apotransferrin (APO) (used as a positive control). Data expressed in % of APO-treated cultures are presented as mean±SEM (n=3). One-way ANOVA followed by Dunnett's post-hoc test. *** p<0.001 vs non-treated cultures.

FIG. 7 depicts histograms showing the anti-pain effects of compounds of formula (Ia) and (C) according to the present invention, compared with morphine. This figure shows the impact of a 3 days-treatment (2 subcutaneous injections/day) with compound of formula (C) (5 mg/kg of animal, 10 mg/kg of animal, 20 mg/kg of animal) or compound of formula (Ia) (10 mg/kg of animal, 20 mg/kg of animal, 40 mg/kg of animal) on formalin-induced pain behavior in male adult mice. Morphine (MOR) is used at 10 mg/kg of animal as single injection. Data expressed in time (sec) spent in licking/bitting behavior are mean values±SEM (n=6). * p<0.05 vs vehicle. One-way ANOVA followed by Bonferroni post-hoc test. Veh=vehicle (placebo).

FIG. 8 depicts histograms showing the anti-pain effects of compounds of formula (Ia) and (C) according to the present invention, compared with morphine. This figure shows the impact of a single subcutaneous injection of compound of formula (C) (10 mg/kg of animal) or compound of formula (Ia) (40 mg/kg of animal) on formalin-induced pain behavior in male adult mice. Morphine (MOR) is used at 10 mg/kg of animal. Data expressed in time (sec) spent in licking/bitting behavior are mean values±SEM (n=6). * p<0.05 vs vehicle. One-way ANOVA followed by Bonferroni post-hoc test. Veh=vehicle (placebo).

FIG. 9 depicts histograms showing the anti-pain effects of compounds of formula (Ia) and (C) according to the present invention, compared with morphine. This figure shows the comparison of the impact of a single subcutaneous injection of compound of formula (C) (10 mg/kg of animal) or compound of formula (Ia) (40 mg/kg of animal) on formalin-induced pain behavior in male and female adult mice. Morphine (MOR) is used at 10 mg/kg of animal. Data expressed in time (sec) spent in licking/bitting behavior are mean values±SEM (n=7). * p<0.05 vs vehicle. One-way ANOVA followed by the Bonferroni post-hoc test. Veh=vehicle (placebo).

FIG. 10 is an histogram showing the anti-inflammatory effects of the compound of formula (Ib) according to the present invention in cultivated microglial cells challenged with LPS. This figure shows the anti-inflammatory effects of the compound of formula (Ib) in LPS-activated mouse microglial cells. Results expressed as means±SEM (n=3-6) are given in pg/mL. **** p<0.0001 vs. non-treated cells; ^{####}p<0.0001 vs. LPS. One-way ANOVA followed by Bonferroni's post-hoc test.

### EXAMPLES

The present invention is further illustrated by the following examples.

### Example 1

### Synthesis of the Compound of Formula (Ia) According to the Present Invention

### Materials and Methods

In a 500 mL round-bottom flask, doxycycline monohydrate (5.5 g, 11.9 mmol, 1.0 eq) was suspended in water (50 mL) and stirred, followed by the addition of 35% HCl (1.5 mL, 17 mmol, 1.4 equiv) and AcOH (50 mL). After the dissolution of the mixture, zinc dust was added (7.8 g, 119 mmol, 10 eq), and the reaction mixture was stirred at room temperature for 4 hours. The resulting mixture was filtered through a small pad of Celite with acetic acid (AcOH). The organic phase was extracted with CH₂Cl₂, washed with HCl (1M) and brine, dried over MgSO₄, filtered off and concentrated in vacuo. The purification of the residue was performed on an automatic combi flash chromatography [Petroleum ether: Acetone+1% HCOOH; silicagel column Buchi Flashpure 120 g; 0% to 10% acetone over 20 min, then 10% to 11.3% acetone over 5.1 min, then isocratic for 22.7 min, then 11.3 to 20% acetone over 10.4 min (total 58.2 min)].

The reaction scheme is as follows:

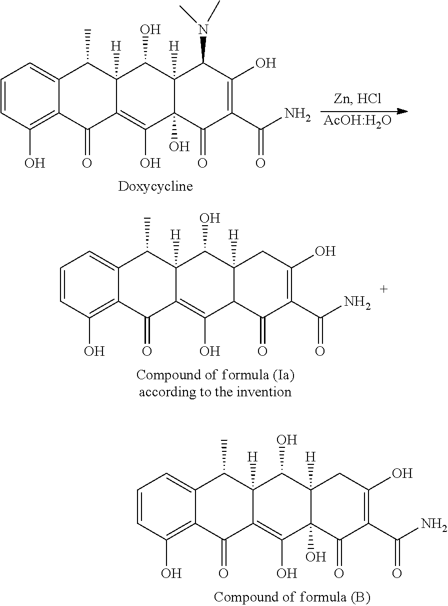

The chemical structure of the obtained compound of formula (Ia) according to the present invention was verified by proton (¹H) and carbon (¹³C) NMR (Nuclear Magnetic Resonance) analysis performed on an NMR spectrometer. Chemical shifts (δ) were expressed in parts per million (ppm). The residual solvent peak was used as reference. The following abbreviations were used to write the ¹H spectrum: s=singlet, d=doublet, t=triplet, q=quadruplet and m=multiplet. The coupling constants (J), expressed in Hz, were determined for doublets, triplets and quadruplets.

### Results

The compound of formula (Ia) according to the invention has been obtained with a yield of 21% by weight relative to the total weight of the product doxycycline and with a purity rate of >95%.

For the compound of formula (Ia) according to the invention:

¹H NMR (400 MHz, Acetone-d₆) δ=18.49 (s, 1H), 14.91 (s, 1H), 12.15 (s, 1H), 9.24 (s, 1H), 7.70 (s, 1H), 7.51 (t, J=7.9 Hz, 1H), 6.84 (d, J=7.5 Hz, 1H), 6.76 (d, J=8.3 Hz, 1H), 4.54 (brd, J=6.6 Hz, 1H), 3.94 (d, J=4.9 Hz, 1H), 3.71 (q, J=7.5 Hz, 1H), 3.35 (ddd, J=10.5, 9.0, 5.0 Hz, 1H), 2.81 (d, J=4.6 Hz, 1H), 2.63 (ddd, J=13.5, 8.7, 4.6 Hz, 1H), 2.44-2.31 (m, 2H), 1.48 (d, J=7.4 Hz, 3H).

¹³C NMR (101 MHz, Acetone-d₆) δ=201.06, 197.50, 191.26, 174.77, 168.87, 163.67, 148.75, 138.28, 120.90, 116.20, 115.53, 103.93, 99.86, 70.98, 49.05, 46.74, 39.82, 37.30, 32.34, 22.96.

The calculated mass of the compound of formula (Ia) according to the invention was of 386.1234 g.mol⁻¹ and the found mass was of 386.1238 g.mol⁻¹ in HRMS (High Resolution Mass spectrum) (HRMS (ESI) m/z: calculated for [M+H]⁺ C₂₀H₁₉NO₇ 386.1234; Found 386.1238).

### Example 2

### Evaluation of the Antiaggregant Effect of the Compound of Formula (Ia) According to the Present Invention

### Materials and Methods

The antiaggregant effect of the compound of formula (Ia) according to the present invention on alpha-synuclein (αS) was evaluated in vitro by measuring the fluorescence intensity of the probe thioflavin T (ThT), a molecule that specifically binds to cross-β structures characteristic of amyloid-like aggregation. The compound of formula (Ia) was dissolved in dimethylsulfoxide to reach a concentration of 50 μM. In this assay, 70 μM of alpha-synuclein was incubated with and without 10 μM, 20 μM and 50 μM of the compound of formula (Ia), under orbital agitation (600 rpm), at 37° C., for 5 days. After the incubation time, Tht was added to each sample and changes in the emission fluorescence spectra (λₑₓ=450 and λₑₘ=48 nm) was monitored using a Horiba Fluoromax CP4 spectrofluorometer.

By using transmission electron microscopy (TEM) we studied the alpha-synuclein aggregation in the presence of 20 μM of the compound of formula (Ia) in the same condition described above. For the samples mounting, 5 μL of a 7 μM alpha-synuclein solution were adsorbed onto a formvar carbon film coated copper grids (Electron Microscopy Sciences) and stained with uranyless.

### Results

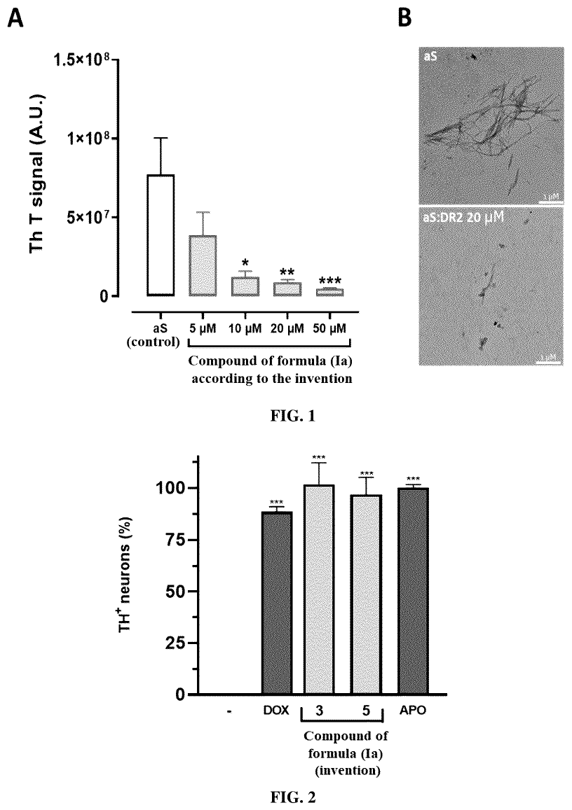

The obtained data, related to changes in the emission fluorescence spectra, show that the compound of formula (Ia) according to the invention significantly inhibits the alpha-synuclein aggregation at 10 μM (p=0.0360), 20 μM (p=0.0077) and 50 μM (p=0.0002) compared to the control (αS) in a dose dependent manner (see FIG. 1A).

Pictures taken from different fields revealed that, in accordance with Tht results (FIG. 1A), the presence of 20 μM of the compound of formula (Ia) according to the invention clearly decreases the amount of alpha-synuclein fibril (FIG. 1B), compared with the control (alpha-synuclein alone) where the typical fibrils morphology can be seen.

### Example 3

### Comparison of the Antimicrobial Activity of the Compound of Formula (Ia) According to the Present Invention with Those of Compounds of the Prior Art

### Materials and Methods

Bacterial strains used in this study were: S. aureus (ATCC25923), E. coli (ATCC25922), E. faecalis (ATCC29212) and P. aeruginosa (PA01). Strains were maintained at −80° C. in 15% (v/v) glycerol for cryoprotection. Bacteria were routinely grown in Mueller-Hinton (MH) broth at 37° C.

The susceptibility of bacterial strains to antibiotics and compounds was determined in microplates using the standard broth dilution method in accordance with the recommendations of the Comité de 1′Antibiogramme de la Société Française de Microbiologie (CA-SFM). (Members of the SFM Antibiogram Committee, R. Int. J. Antimicrob. Agents 2003, 21, 364). Briefly, the Minimal Inhibitory Concentrations (MICs) were determined with an inoculum of 10⁵ CFU in 200 μL of MH broth containing two-fold serial dilutions of each drug. The MIC was defined as the lowest concentration of drug that completely inhibited visible growth after incubation for 18 h at 37° C. To determine all MICs, the measurements were independently repeated in triplicate.

The compound of formula (Ia) according to the invention was tested against clinical isolates Gram-bacteria (P. aeruginosa, E. coli) and Gram+ bacteria (S. aureus, E. faecalis). Minimum inhibitory concentrations (MIC) were tentatively determined and compared to known antibiotic tetracyclines (doxycycline, demeclocycline, chlortetracycline) and to the compound of formula (B) disclosed in Example 1 of the present application. The test was reproduced twice and the average MIC were calculated.

### Results

The results are presented in the following table:

TABLE 1

| | average MIC (μM) | | | |
|---|---|---|---|---|
| Tested | P. aeruginosa | E. coli ATCC | S. aureus | E. faecalis |
| compounds | PA01 | 25922 | ATCC 25923 | ATCC 29212 |
| Compound of | >200 | >200 | >200 | >200 |
| formula (Ia) | | | | |
| according to the | | | | |
| invention | | | | |
| Compound of | >2000 | 200 | 25 | — |
| formula (B) | | | | |
| Doxycycline | 64 | 3.125 | 0.4 | 0.4 |
| Demeclocycline | 32 | 3.125 | 0.4 | 0.4 |
| Chlortetracycline | 32 | 6.25 | 1.6 | 1.6 |

As it can be seen in Table 1, surprisingly and interestingly, the compound of formula (la) according to the invention did not show any activity against Gram+ and Gram− bacteria, up to 200 mg/mL, and was less active than the compound of formula (B) on S. aureus.

### Example 4

### Comparison of the Neuroprotection of the Compound of Formula (Ia) According to the Present Invention with Those of a Compound of the Prior Art

### Materials and Methods

In primary cultures of dopaminergic neurons, TH+ neurons suffer from oxidative stress, leading to the death of the neurons after a few days. When cultured in the presence of a positive reference (apotransferrin), the dopaminergic neurons survive. The neuroprotective effects of 10 μM of doxycycline (DOX) and of 3 μM or 5 μM of the compound of formula (Ia) according to the present invention were evaluated, compared to the effect of 100 μg/mL of apotransferrin (APO).

### Results

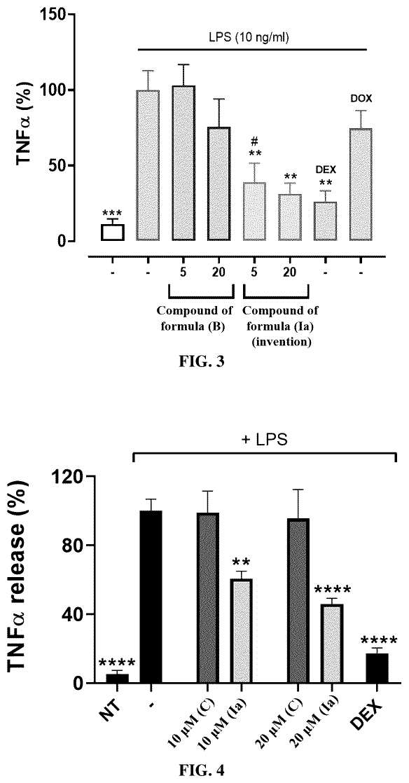

The results are presented in FIG. 2. As it can be seen in FIG. 2, compared to doxycycline, which shows a neuroprotective effect at 10 μM, the compound of formula (Ia) according to the present invention was highly protective at only 3 μM and 5 μM.

### Example 5

### Comparison of the Anti-Inflammatory Effect of the Compound of Formula (Ia) According to the Present Invention With Those of Compounds of the Prior Art

### Materials and Methods

Microglia is in charge of the protection of the neurons. However, under stress conditions, microglia cells are responsible of inflammatory phenomena, leading eventually to the death of the neurons.

The anti-inflammatory effects of 5 μM and 20 μM of compound of formula (B) presented in Example 1, of 5 μM and 20 μM of compound of formula (Ia) according to the present invention and of 50 μM of doxycycline (DOX) were evaluated, compared to the effect of 2.5 μM of dexamethasone (DEX).

### Results

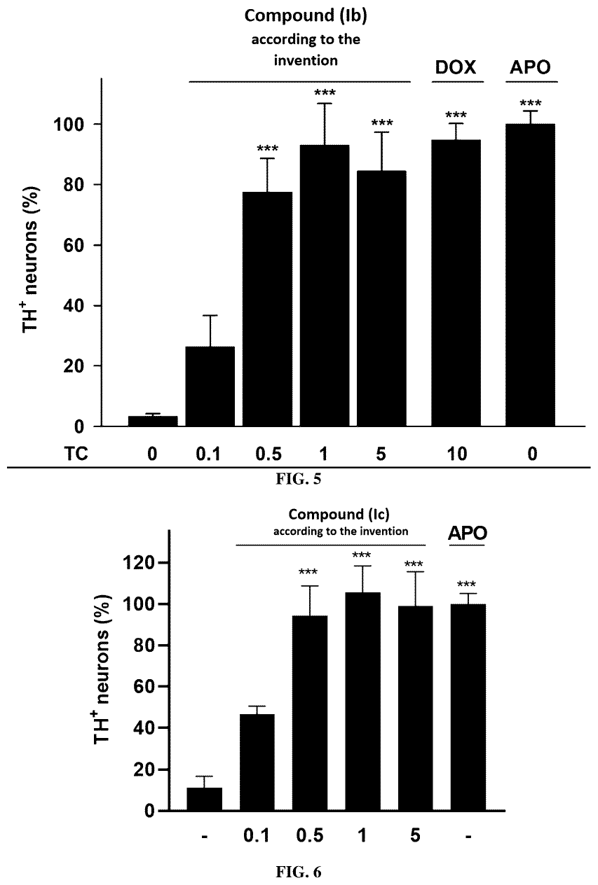

The results are presented in FIG. 3. As it can be seen in FIG. 3, the compound of formula (Ia) counteracts inflammatory process (generated by LPS) in an efficient manner, superior to both doxycycline and compound of formula (B).

### Example 6

### Comparison of the Anti-Inflammatory Effect of the Compound of Formula (Ia) According to the Present Invention With Those of Compounds of the Prior Art

### Materials and Methods

Microglia is in charge of the protection of the neurons. However, under stress conditions, microglia cells are responsible of inflammatory phenomena, leading eventually to the death of the neurons.

The anti-inflammatory effects of 10 μM and 20 μM of compound of formula (C) (“10 μM (C)” and “20 μM (C)”) and of 10 μM and 20 μM of compound of formula (Ia) according to the present invention (“10 μM (Ia)” and “20 μM (Ia)”) were evaluated, compared to the effect of 2.5 μM of dexamethasone (“DEX”).

“Compound of formula (C)” refers to the molecule of the following formula:

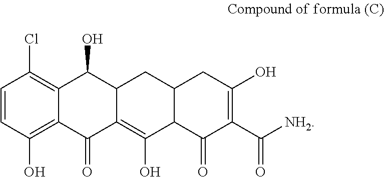 [SMILES: NC(=O)C1=C(O)CC2CC3C(=C(O)C2C1=O)C(=O)c1c(O)ccc(Cl)c1[C@H]3O]

### Results

The results are presented in FIG. 4. As it can be seen in FIG. 4, the compound of formula (Ia) counteracts inflammatory process (generated by LPS) in an efficient manner, whereas compound of formula (C) was unable to counteract the inflammatory process.

### Example 7

### Comparison of the Properties of the Compound of Formula (Ia) According to The Present Invention With Those of Compounds of the Prior Art

### Materials and Methods

The properties of tetracycline derivatives, including the compound of formula (Ia) according to the present invention, were compared regarding the neuroprotection, anti-inflammatory effects, anti-aggregant activity, and antibiotic activity. The properties were evaluated as described herein above.

The following compounds were tested: compound of formula (Ia) according to the invention, compound of formula (C) as disclosed in Example 6 of the present application, compound of formula (D), compound of formula (E), doxycycline, compound of formula (B) as disclosed in Example 1 of the present application, chlortetracycline, compound of formula (F), compound of formula (G), and compound of formula (H).

“Compound of formula (D)” refers to the molecule of the following formula:

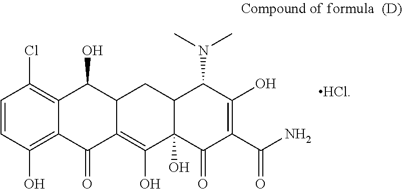 [SMILES: CN(C)[C@@H]1C(O)=C(C(N)=O)C(=O)[C@@]2(O)C(O)=C3C(=O)c4c(O)ccc(Cl)c4[C@@H](O)C3CC12]

“Compound of formula (E)” refers to the molecule of the following formula:

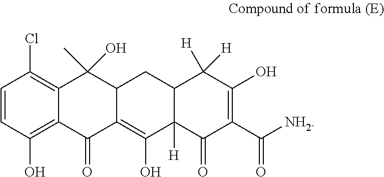 [SMILES: CC1(O)c2c(Cl)ccc(O)c2C(=O)C2=C(O)C3C(=O)C(C(N)=O)=C(O)CC3CC21]

“Compound of formula (F)” refers to incyclinide (CMT-3, COL-3), which has the CAS number 15866-90-7 and the following formula:

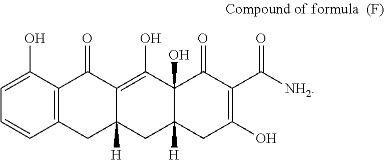 [SMILES: NC(=O)C1=C(O)C[C@@H]2C[C@@H]3Cc4cccc(O)c4C(=O)C3=C(O)[C@]2(O)C1=O]

“Compound of formula (G)” refers to the molecule of the following formula:

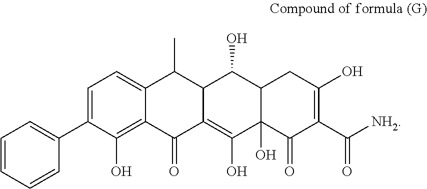 [SMILES: CC1c2ccc(-c3ccccc3)c(O)c2C(=O)C2=C(O)C3(O)C(=O)C(C(N)=O)=C(O)CC3[C@@H](O)C21]

“Compound of formula (H)” refers to the molecule of the following formula:

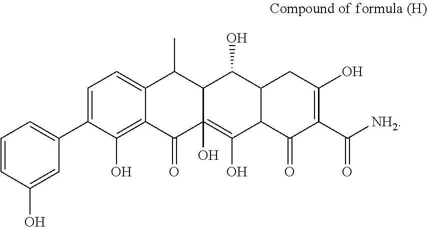

### Results

The summary of the properties of tetracycline derivatives is presented in the following table.

TABLE 2

| | | Anti | Anti | |
|---|---|---|---|---|
| | | inflammatory | aggregant | |
| Tetracycline | Neuroprotection* | effects** | activity at | Antibiotic |
| derivative | (EC 50 μM) | (IC 50 in μM) | 20 μM | activity |
| Compound of | 1.58 | yes | yes | null |
| formula (Ia) | | | | |
| according to the | | | | |
| invention | | | | |
| Compound of | 0.81 | no | yes | low^{#} |
| formula (C) | | | | |
| Compound of | 1.84 | no | yes | high^{##} |
| formula (D) | | | | |
| Compound of | 2.01 | no | yes | low |
| formula (E) | | | | |
| Doxycycline | 5.2 | 45 | yes | high |
| Compound of | no | no | yes | low |
| formula (B) | | | | |
| Chlortetracycline | no | 1 | yes | high |
| Compound of | no | 8 | yes | Significant^{###} |
| formula (F) | | | | |
| Compound of | no | 8 | yes | low |
| formula (G) | | | | |
| Compound of | no | no | no | low |
| formula (H) | | | | |

EC50s, IC50s determined graphically, *Rescue of dopamine neurons from spontaneous oxidative stress; ** TNF α release inhibition under LPS exposure, ^{#}Weak antibiotic activity on only 1 of the 3 bacterial strains (i.e. S aureus). ##Strong antibiotic activity on all 3 bacterial strains. ^{###}Significant antibiotic activity on two bacterial strains.

As shown herein above, the compound of formula (Ia) according to the present invention presents advantageous properties compared to the other tetracycline derivatives. In particular, the compound of formula (Ia) according to the present invention has neuroprotective, anti-inflammatory and anti-aggregant effects, and no antibiotic activity.

### Example 8

### Comparison of the Neuroprotection of the Compound of Formula (Ib) According to the Present Invention With Those of a Compound of the Prior Art

### Materials and Methods

Midbrain mouse cultures were subjected to a maturation step until day in vitro (div) 7, and then gradually exposed to phenol red formulated DMEM/Nutrient mixture F12 Ham supplemented with 20 μg/mL of insulin and 2 μM of the NMDA receptor blocker MK-801. Under these conditions, which favor low-level, sustained oxidative insults, and lead ultimately to dopamine cell death, treatments with the compound of formula (Ib) according to the invention (at a concentration ranging from 0.1 to 5 μM) were performed or not, as well as treatments with the reference tetracycline antibiotic doxycycline (DOX; 10 μM) and the glycoprotein apotransferrin (APO; 100 μg/mL), the latter being used as reference neuroprotectant. At div14, cultures were fixed, and then processed for tyrosine hydroxylase (TH) immunocytochemistry to estimate the survival of dopamine neurons.

### Results

The results are presented in FIG. 5. As it can be seen in FIG. 5, compared to doxycycline, which shows a neuroprotective effect at 10 μM, the compound of formula (Ib) according to the present invention was highly protective at only 0.5 μM, 1 μM and 5 μM. Thus, these results show that the compound of formula (Ib) according to the present invention efficiently protects dopamine neurons from degeneration.

In addition, the effect of the compound of formula (Ib) according to the present invention is reproduced by the glycoprotein apotransferrin, a molecule with iron chelation capacity, suggesting that said compound of formula (Ib) according to the present invention operates by preventing the deleterious consequences of a Fenton-type reaction generating hydroxyl radicals and as a result oxidative degradation of lipids. Overall, these results confirm that the compound of formula (Ib) according to the present invention is of potential value against low-level oxidative stress damage that develops in particular chronically in the course of PD neurodegeneration.

### Example 9

### Comparison of the Antimicrobial Activity of the Compound of Formula (Ib) According to the Present Invention With Those of Compounds of the Prior Art

### Materials and Methods

Bacterial strains used in this study were: S. aureus (ATCC25923), E. coli (ATCC28922), E. faecalis (ATCC29212) and P. aeruginosa (PA01). The minimum inhibitory concentrations (MICs) were estimated using standardized protocols (Mawabo et al, J. Infect. Public Health 2015).

### Results

The results are presented in the following table:

TABLE 2

| | average MIC (μM) | | | |
|---|---|---|---|---|
| | P. aeruginosa | E. coli ATCC | S. aureus | E. faecalis |
| Tested compounds | PA01 | 28922 | ATCC 25923 | ATCC 29212 |
| Compound of formula | >200 | >200 | 100 | 100 |
| (Ib) according to the | | | | |
| invention | | | | |
| Oxytetracycline | 50 | 3.125 | 3.125 | 3.125 |

As it can be seen in Table 2, surprisingly and interestingly, Data show that oxytetracycline exerts antimicrobial effects against 3 of the 4 test bacterial strains whereas the compound of formula (Ib) according to the invention is effective against none of them.

### Example 10

### Analgesic Effect of the Compound of Formula (Ia) According to the Present Invention and of the Compound of Formula (C) Described in Example 6

### Materials and Methods

The analgesic effects of the compound of formula (C) described in Example 6 (administered at doses of 5 mg/kg of animal, 10 mg/kg of animal and 20 mg/kg of animal) and the compound of formula (Ia) according to the present invention (administered at doses of 10 mg/kg of animal, 20 mg/kg of animal, and 40 mg/kg of animal) were tested using the formalin-induced nociceptive pain model (as disclosed in the publication Lopes DM, Cater HL, Thakur M et al. “A refinement to the formalin test in mice [version 2; peer review: 2 approved]” F1000Research 2019, 8:891).

### Results

The results showed that the non-antibiotic tetracyclines of formula (Ia) and of formula (C) reduce pain-related responses in mice subjected to the formalin test.

In particular, it was found that a sub-chronic treatment regimen (6 subcutaneous daily injections) of male adult C57/BL6 mice with 5 mg/kg of the compound of formula (C) was highly effective in reducing nociceptive pain in phase 1 and 2 of the test. These effects were not different from those of morphine (10 mg/kg) administered similarly. The compound of formula (Ia) was also effective and exerted analgesic effects between 20and 40 mg/kg in phases 1 and 2 of the test. Interestingly, a single injection of the compound of formula (C) (10 mg/kg) prior to formalin injection resulted in robust anti-nociceptive effects in both phases 1 and 2 of the test, and this effect was not different from that of morphine (10 mg/kg). A single injection of the compound of formula (Ia) (40 mg/kg) prior to formalin injection exerted anti-nociceptive effects as well in both phases. Noticeably, male mice exhibited a better analgesic response to a single injection of the compound of formula (C) (10 mg/kg), while females responded more effectively to the corresponding treatment with the compound of formula (Ia) (40 mg/kg) in phase 2 of the formalin test; this suggests a sex-dependent analgesic effect of the compound of formula (C) and the compound of formula (Ia).

Thus, the results showed that 12a-deoxytetracycline have anti-nociceptive effects.

### Example 11

### Capacity to Penetrate the Brain of the Compound Of Formula (Ia) According to the Present Invention and of the Compound of Formula (C) Described in Example 6

### Materials and Methods

Adult Swiss mice received a single subcutaneous injection of 40 mg/kg of the compound of formula (Ia) according to the present invention, the compound of formula (C), or doxycycline diluted in saline with 5% DMSO and 5% Tween 80 (doxycycline was used as a reference tetracycline). The mice were sacrificed 30 min, 1 h, 8 h, and 24 h after treatment. After sacrifice, brain and serum samples were collected and processed at each time point for tetracycline dosage, using an UHPLC system coupled with a triple quadrupole mass spectrometer LCMS-8030 (Shimadzu Corporation, Kyoto, Japan).

### Results

The brain-to-plasma ratio calculated from the area under the concentration time curves in the brain and plasma were 0.27±0.13, 0.21±0.05, and 0.11±0.03 (n =3) for the compound of formula (C), the compound of formula (Ia), and doxycycline, respectively, suggesting that the compound of formula (C) and the compound of formula (Ia) penetrate the brain better than doxycycline.

### Example 12

### Inhibition of the Phosphorylation of Protein Tau by the Compound of Formula (Ib) According to the Present Invention and the Compound of Formula (C) Described in Example 6

### Materials and Methods

A culture model of cortical neurons from mouse embryonic brain was used, in which neuronal cells were subjected to a maturation step until day in vitro (div) 7, before being gradually exposed to phenol red formulated DMEM/Nutrient mixture F12 Ham supplemented with 20 μg/mL of insulin, and 2 μM of the NMDA receptor blocker MK-801 (DF12i).

### Results

Under these conditions, which promote sustained low-level oxidative insults because of the presence of small amounts of catalytic iron in DF12i medium, it was found that a significant proportion of Microtubule-Associated Protein-2+ neurons exhibit high levels of p-tau (AT8 immunosignal) (p-tau=phosphorylated tau protein at Ser202, Thr205 residues) in their somas and neuritic extensions when degeneration is in progress. Precisely, after 4 days of exposure to DF12i, between 10-15% of surviving neurons show a very strong AT8 immunosignal in their somas while only 1 to 1.5% of neuronal cell bodies are found immunopositive under conditions where oxidative stress is repressed by treatment with bovine apotransferrin (APO; 100 g/mL, Sigma Aldrich), a glycoprotein with iron chelating properties. Likewise, overexpression of p-tau in cortical cell bodies is reduced by the two non-antibiotic tetracyclines compound of formula (C) (3 M) and compound of formula (Ib) (3 M): see the following Table:

TABLE
Impact of compound of formula (Ib) and compound of formula (C) on
the expression of pathological tau in cultured cortical neurons

| | Reduction of the % |
|---|---|
| Treatment | of p-tau+ somas |
| none | − |
| compound of formula (C) (3 μM) | +++ |
| compound of formula (Ib) (3 μM) | +++ |
| APO 100 μg/mL | +++ |

p-tau+ (AT8 antibody) cell bodies were estimated in mature mouse cortical cultures exposed between div 7-11 to DF12i before being fixed with formaldehyde, and processed for immunofluorescence detection and microscopical examination.

In the above table, a favorable response, synonymous with a decrease in the number of p-tau+ cell bodies in the presence of the treatments of interest, is designated by a sign “+”, “++”, or “+++” (+++=reference level). Conversely, an unfavorable response is represented by a sign “−”, synonymous with an elevation of the number of p-tau+ cell bodies. Compound of formula (Ib) and compound of formula (C) reduced the number of p-tau+ soma (probably by limiting the impact of oxidative insults). APO reduced the number of p-tau+ cell bodies (probably by its capacity of chelating iron, and therefore preventing iron-mediated oxidative stress).

In conclusion, there is an inhibition of the pathological phosphorylation of tau by the compound of formula (Ib) according to the present invention and the compound of formula (C) described in Example 6. The phosphorylation of tau is the first step to its aggregation. Indeed, in Alzheimer's disease and related disorders called tauopathies, tau becomes hyperphosphorylated and mislocalized, which can contribute to its aggregation and toxicity. Thus, the compound of formula (Ib) according to the present invention and the compound of formula (C) are of therapeutic interest in the treatment of diseases in which the protein tau is involved (in particular Alzheimer's disease and related disorders called tauopathies).

### Example 13

### Comparison of the Neuroprotection of the Compound of Formula (Ic) According to the Present Invention with Those of a Compound of the Prior Art

### Materials and Methods

Midbrain mouse cultures were subjected to a maturation step until day in vitro (div) 7, and then gradually exposed to phenol red formulated DMEM/Nutrient mixture F12 Ham supplemented with 20 μg/mL of insulin and 2 μM of the NMDA receptor blocker MK-801. Under these conditions, which favor low-level, sustained oxidative insults, and lead ultimately to dopamine cell death, treatments with the compound of formula (Ic) according to the invention (at a concentration ranging from 0.1 to 5 μM) were performed or not, as well as treatments with the glycoprotein apotransferrin (APO; 100 μg/mL), the latter being used as reference neuroprotectant. At div14, cultures were fixed, and then processed for tyrosine hydroxylase (TH) immunocytochemistry to estimate the survival of dopamine neurons.

### Results

The results are presented in FIG. 6. As it can be seen in FIG. 6, the compound of formula (Ic) according to the present invention was highly protective at only 0.5 μM, 1 μM and 5 μM. Thus, these results show that the compound of formula (Ic) according to the present invention efficiently protects dopamine neurons from degeneration in a concentration-dependent manner.

In addition, the compound of formula (Ic) according to the invention does not show any antimicrobial effect against bacteria.

Furthermore, the compound of formula (Ic) according to the invention has anti-inflammatory effects vis-à-vis 10 ng/mL LPS, at 5 μM, 25 μM and 50 μM.

### Example 14

### Anti-Pain Effects of Compounds of Formula (Ia) and (C) According to the Present Invention

Materials and Methods

To test the anti-pain effects of compounds (Ia) and (C) according to the present invention, a formalin pain test was performed, which consists in subcutaneously injecting 50 μl of a 5.0% formalin solution into the right hind paw of adult C57/BL6 mice (Taylor and Basbaum, J Pain, 2000) before scoring pain behavior. The intensity of pain was estimated by recording the cumulative time (sec) during which animals develop a licking/bitting behavior in response to formalin injection. This response is divided into a transient early (Phase I) phase followed by a more prolonged late (Phase II) phase. Pain scoring is performed by experimenters blinded to test treatments using a video recording of formalin-induced pain behavior.

### Results

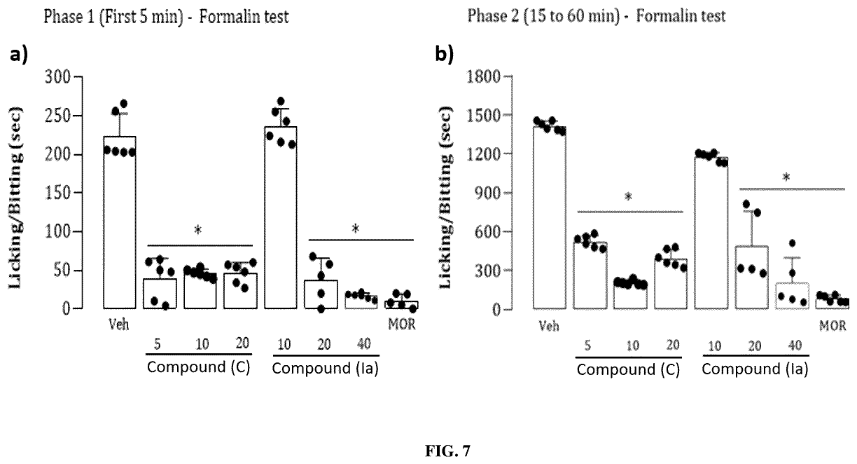

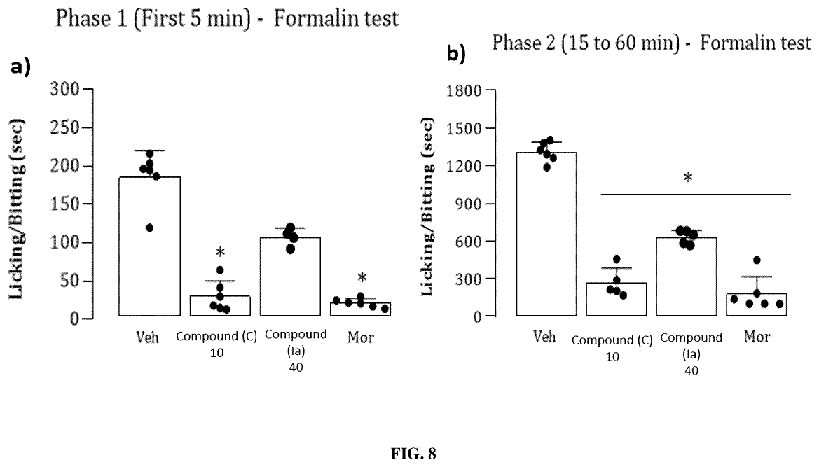

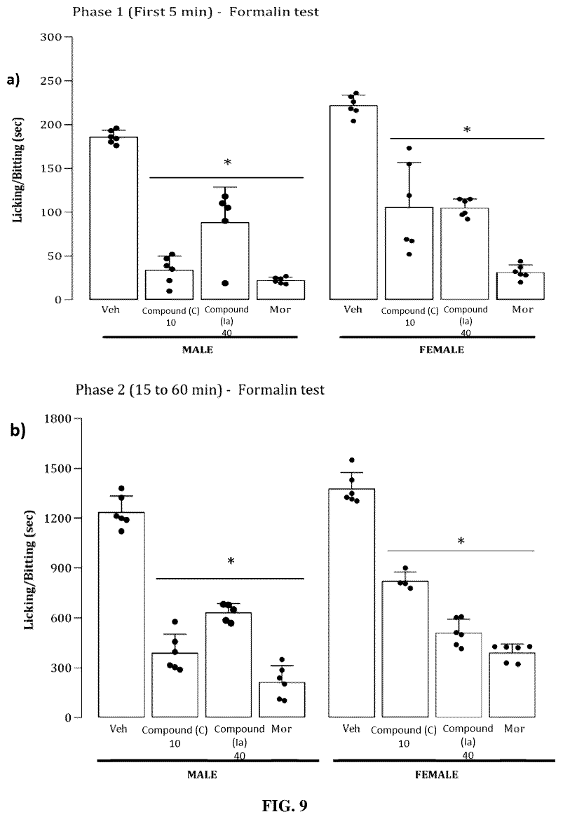

The results are presented in FIG. 7, FIG. 8 and FIG. 9. As it can be seen, both compounds (Ia) and (C) according to the present invention showed anti-pain effects.

### Example 15

### Anti-inflammatory Effect of the Compound of Formula (Ib) According to the Present Invention

### Materials and Methods

Primary mouse microglial cells cultivated in Dulbecco's Modified Eagle Medium (DMEM) with no serum and 15 mM Hepes were challenged with lipopolysaccharide (LPS, 10 ng/ml) for 24 hours in the presence or not of the compound of formula (Ib) according to the present invention at concentrations of 1 μM, 5 μM, 25 μM and 50 μM. After that, the release of the proinflammatory cytokine TNF-a was estimated with an ELISA kit (ThermoFisher Scientific).

### Results

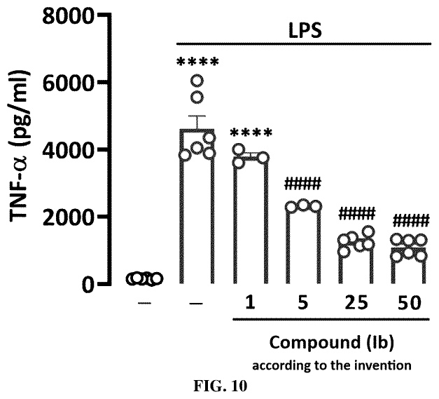

The results are presented in FIG. 10. As it can be seen in FIG. 10, the results show that the compound of formula (Ib) according to the present invention reduces significantly the proinflammatory effects of LPS at concentrations between 5 μM to 50 μM.

1-23. (canceled)

24. A method of treating or preventing a disease selected from the group consisting of: amyloidosis, pain, neurodegenerative diseases and neuroinflammatory diseases in a subject, comprising administering to the subject a therapeutically effective amount of a compound of formula (I) or a pharmaceutically acceptable salt, solvate or mixtures thereof,
wherein said formula (I) is: 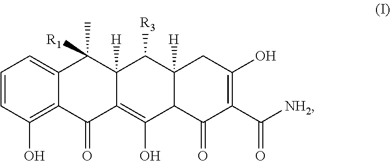
and
wherein R₁ and R₃ are each independently selected from the group consisting of a hydrogen atom and a hydroxyl group.

25. The method according to claim 24, wherein the compound of formula (I) is the compound of formula (Ibis) or a pharmaceutically acceptable salt, solvate or mixtures thereof, wherein said formula (Ibis) is: 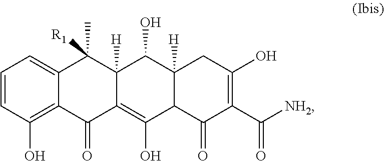
wherein R₁ is a hydrogen atom or a hydroxyl group.

26. The method according to claim 24, wherein the compound of formula (I) is the compound of formula (Ia) or a pharmaceutically acceptable salt, solvate or mixtures thereof, wherein said formula (Ia) is:

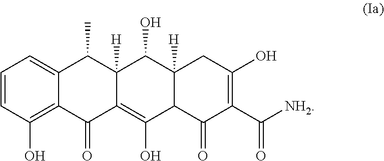 [SMILES: C[C@H]1c2cccc(O)c2C(=O)C2=C(O)C3C(=O)C(C(N)=O)=C(O)C[C@@H]3[C@@H](O)[C@@H]21]

27. The method according to claim 24, wherein the compound of formula (I) is the compound of formula (Ib) or a pharmaceutically acceptable salt, solvate or mixtures thereof, wherein said formula (Ib) is:

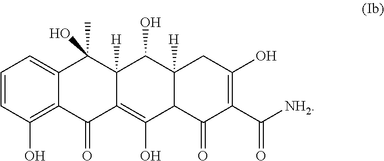 [SMILES: C[C@@]1(O)c2cccc(O)c2C(=O)C2=C(O)C3C(=O)C(C(N)=O)=C(O)C[C@@H]3[C@@H](O)[C@@H]21]

28. The method according to claim 24, wherein the compound of formula (I) is the compound of formula (Ic) or a pharmaceutically acceptable salt, solvate or mixtures thereof, wherein said formula (Ic) is:

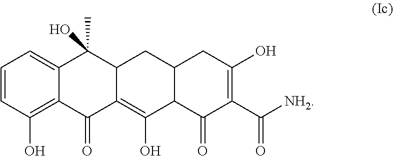 [SMILES: C[C@@]1(O)c2cccc(O)c2C(=O)C2=C(O)C3C(=O)C(C(N)=O)=C(O)CC3CC21]

29. The method according to claim 24, wherein the disease is selected from the group consisting of: amyloidosis, neuropathic pain, neurodegenerative diseases and neuroinflammatory diseases.

30. The method according to claim 24, wherein said neurodegenerative diseases and neuroinflammatory diseases are selected from the group consisting of: Parkinson's disease, diffuse Lewy body disease, multiple system atrophy, Alzheimer's disease, prion disease, multiple sclerosis, limbic-predominant age-related TDP-43 encephalopathy (LATE), and Huntington's disease.

31. The method according to claim 24, wherein said neurodegenerative disease is Parkinson's disease or Alzheimer's disease.

32. A pharmaceutical composition comprising a compound of formula (I) or pharmaceutically acceptable salt, solvate or mixtures thereof, wherein said formula (I) is: 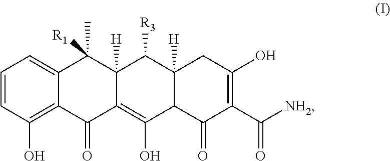
wherein R₁ and R₃ are each independently selected from the group consisting of a hydrogen atom and a hydroxyl group, and at least one pharmaceutically acceptable excipient.

33. The pharmaceutical composition according to claim 32, wherein the compound of formula (I) is the compound of formula (Ibis) or a pharmaceutically acceptable salt, solvate or mixtures thereof, wherein said formula (Ibis) is: 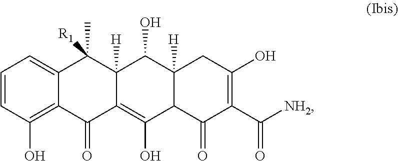
wherein R₁ is a hydrogen atom or a hydroxyl group.

34. The pharmaceutical composition according to claim 32, wherein the compound of formula (I) is the compound of formula (Ia) or a pharmaceutically acceptable salt, solvate or mixtures thereof, wherein said formula (Ia) is:

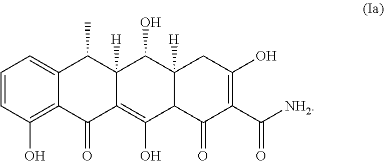 [SMILES: C[C@H]1c2cccc(O)c2C(=O)C2=C(O)C3C(=O)C(C(N)=O)=C(O)C[C@@H]3[C@@H](O)[C@@H]21]

35. The pharmaceutical composition according to claim 32, wherein the compound of formula (I) is the compound of formula (Ib) or a pharmaceutically acceptable salt, solvate or mixtures thereof, wherein said formula (Ib) is:

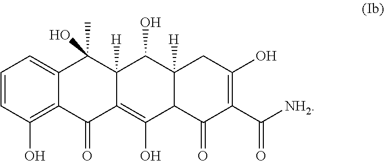 [SMILES: C[C@@]1(O)c2cccc(O)c2C(=O)C2=C(O)C3C(=O)C(C(N)=O)=C(O)C[C@@H]3[C@@H](O)[C@@H]21]

36. The pharmaceutical composition according to claim 32, wherein the compound of formula (I) is the compound of formula (Ic) or a pharmaceutically acceptable salt, solvate or mixtures thereof, wherein said formula (Ic) is:

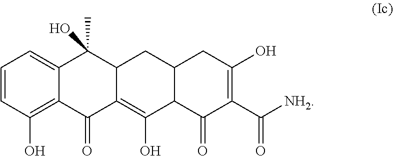 [SMILES: C[C@@]1(O)c2cccc(O)c2C(=O)C2=C(O)C3C(=O)C(C(N)=O)=C(O)CC3CC21]

37. The pharmaceutical composition according to claim 32, wherein said at least one pharmaceutically acceptable excipient is selected from the group consisting of: microcrystalline cellulose, magnesium stearate, povidone, hydrogenated castor oil, colloidal anhydrous silica, sodium carboxymethyl starch and combinations thereof.

38. The pharmaceutical composition according to claim 32, for use as a medicament.

39. A method of treating or preventing a disease selected from the group consisting of: amyloidosis, pain, neurodegenerative disease, and neuroinflammatory disease comprising administering to the subject a therapeutically effective amount of the pharmaceutical composition according to claim 32.

40. The method according to claim 39, wherein said pharmaceutical composition is administered to the subject by a route selected from the group consisting of: oral route, intraspinal route, intraarterial route, intravenous route, intramuscular route and subcutaneous route.

41. A method of treating pain in a subject, comprising administering a therapeutically effective amount of a 12a-deoxytetracycline compound to the subject.

42. The method according to claim 41, wherein said 12a-deoxytetracycline is selected from the group consisting of:

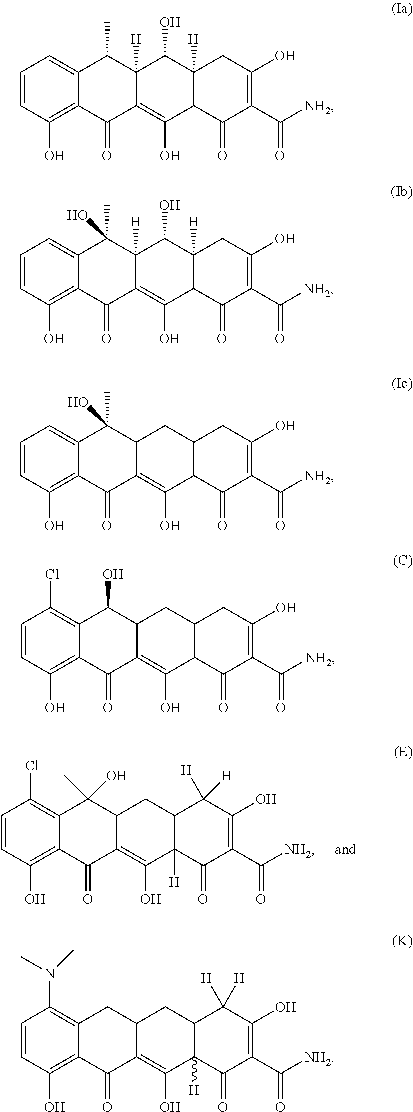 [SMILES: C[C@H]1c2cccc(O)c2C(=O)C2=C(O)C3C(=O)C(C(N)=O)=C(O)C[C@@H]3[C@@H](O)[C@@H]21 ; C[C@@]1(O)c2cccc(O)c2C(=O)C2=C(O)C3C(=O)C(C(N)=O)=C(O)C[C@@H]3[C@@H](O)[C@@H]21 ; C[C@@]1(O)c2cccc(O)c2C(=O)C2=C(O)C3C(=O)C(C(N)=O)=C(O)CC3CC21 ; NC(=O)C1=C(O)CC2CC3C(=C(O)C2C1=O)C(=O)c1c(O)ccc(Cl)c1[C@H]3O ; CC1(O)c2c(Cl)ccc(O)c2C(=O)C2=C(O)C3C(=O)C(C(N)=O)=C(O)CC3CC21 ; CN(C)c1ccc(O)c2c1CC1CC3CC(O)=C(C(N)=O)C(=O)C3C(O)=C1C2=O]

43. The method according to claim 41, wherein said pain is selected from the group consisting of: neuropathic pain, nociceptive pain and centralized pain.

44. The method according to claim 41, wherein said pain is a nociceptive pain induced by a disease selected from the group consisting of: osteoarthritis, rheumatoid arthritis and cancer.

45. The method according to claim 41, wherein said pain is a neuropathic pain induced by diabetic neuropathy.

46. The method according to claim 41, wherein said pain is a centralized pain induced by a disease selected from the group consisting of: fibromyalgia, irritable bowel syndrome and tension headaches.
